(19) United States
(12) Patent Application Publication
(10) Pub. No.: US 2026/0176699 A1
(43) Pub. Date: Jun. 25, 2026

(54) METHODS OF IDENTIFYING AND TREATING INDIVIDUALS WITH ELEVATED CANCER RISK

(71) Applicant: AMAZON TECHNOLOGIES, INC., Reno, NV (US)

(72) Inventors: Haibao Tang, Cupertino, CA (US); Frank Wilhelm Schmitz, Seattle, WA (US); David Heckerman, Bellevue, WA (US); Melanie Walker, Seattle, WA (US); Neeraj Sood, Los Angeles, CA (US); Antje Heit, Seattle, WA (US); Layne Christopher Price, Seattle, WA (US); Anta Imata Safo, New York, NY (US); Samuel Anthony Danziger, Seattle, WA (US); Alena Harley, Los Gatos, CA (US); Beshoy Sarkis, Bothell, WA (US); Sean Michael Stockwell, Seattle, WA (US); Brandon Yacullo Hoane, Seattle, WA (US)

(21) Appl. No.: 18/996,073

(22) PCT Filed: Dec. 13, 2024
(86) PCT No.: PCT/US2024/059930

§ 371 Date: Jan. 17, 2025

Related U.S. Application Data
(60) Provisional application No. 63/609,751, filed on Dec. 13, 2023.
(60) Provisional application No. 63/570,370, filed on Mar. 27, 2024.

(51) Int. Cl. C12Q 1/6886; C12Q 1/6869; C12Q 1/6881

(52) U.S. Cl. CPC C12Q 1/6886; C12Q 1/6869; C12Q 1/6881; C12Q 2600/106; C12Q 2600/118

(57) ABSTRACT

Disclosed herein are methods of treating an individual at risk for incidence or recurrence of cancer in need thereof. Methods can include the steps of identifying an individual at risk for incidence or recurrence of cancer based on a risk stratification parameter; analyzing a biological sample from using a multi-cancer detection (MCD) test to yield an MCD test result; and based on the MCD test result and optionally the risk stratification parameter, administering a neoantigen immunogenic composition to the individual in need thereof. Methods can include the steps of sequencing and analyzing of the biological sample from the individual or a new biological sample from the individual to identify neoantigens to be included in the neoantigen immunogenic composition; and analyzing a second biological sample from the individual at risk for incidence or recurrence of cancer using a second multi-cancer detection (MCD) test to determine efficacy of the neoantigen immunogenic composition.

### CROSS-REFERENCE TO RELATED APPLICATIONS

The present application is a United States National Stage Application of International Patent Application No. PCT/US2024/059930 filed on Dec. 13, 2024, which claims benefit of U.S. Provisional Application No. 63/609,751, filed on Dec. 13, 2023 and U.S. Provisional Application No. 63/570,370, filed Mar. 27, 2024, the entire contents of each of which are incorporated herein by reference.

### BACKGROUND

Early detection is a cornerstone of positive prognosis and successful cancer treatment of neoplastic diseases (e.g., cancers). Combination of early detection and improved treatments for cancer has led to an increase in five-year relative survival rates over the past several decades. For example, the five-year relative survival rates for women with breast cancer have improved from approximately 75% between 1975-1977 to 90% between 2003-2009. S. Saadatmand, et al. Influence of tumour stage at breast cancer detection on survival in modern times: population based study in 173 797 patients BMJ (2015), 351, 1-9.

Early detection methods have recently been bolstered by the development and implementation of multi-cancer detection tests (MCD tests, also known as multi-cancer early detection (MCED) tests). MCD tests take advantage of recent advances in the analysis of the cancer genome, epigenome, transcriptome, metabolome, and proteome of biological samples from an individual. C. E. Guerra, et al. Multi-Cancer Early Detection: The New Frontier in Cancer Early Detection. Ann. Rev. Med. (2024) 75, 67-81. Although early detection methods (e.g., MCD tests) are advancing at a rapid rate, the conventional standard of care often dictates a “watch and wait” approach of delaying treatment until a cancer diagnosis has been validated, a tumor has been located, or the cancer has advanced in stage. Delays in treatment can cause psychological distress, such as anxiety and/or depression, for the individual. D. McCaughan, et al. Patient perspectives of ‘Watch and Wait’ for chronic haematological cancers: Findings from a qualitative study. Eur. J. Oncol. Nurs. (2023) 65, 102349, 1-9. The negative impact of psychological distress on the quality of life of the individual can outweigh any side effects associated with the conventional standard of care treatments and procedures. “‘While people are often initially relieved that they do not have to immediately undergo treatment and face the potential side effects and life disruptions associated with treatment, a watch-and-wait approach can be stressful for some because they don't feel like they are actively ‘doing something’ about their cancer . . . ’” “‘Even if they aren't receiving treatment, the fear of the unknown and a sense of lack of control can result in feelings of helplessness, anxiety or depression.’” R. Tompa, The cancer waiting game: When the recommended treatment is nothing. Fred Hutch News Service. Oct. 11, 2016, https://www.fredhutch.org/en/news/center-news/2016/10/cancer-waiting-game-when-the-recommended-treatment-is-watch-and-wait.html #:˜:text-%E2%80%9CEven%20if%20they%20aren't,helplessness%2C%20anxiety%2 0or%20depression.%E2%80%9D, accessed Feb. 26, 2024 (Quoting Dr. Jesse Fann, director of psychiatry and psychology services at Seattle Cancer Care Alliance). Clearly, “watch and wait” has the potential to negatively affect the mood and mental health status of the individual. Furthermore, delay in treatment may negatively affect the likelihood of successful treatment of the cancer.

For at least these reasons, there is a pressing need for new methods of treating individuals at high or elevated risk for developing cancer or at high risk of cancer recurrence with treatments that can delay, prevent, or cure cancer. Useful methods would have few or no side effects to allow for the early intervention in individuals at risk for incidence or recurrence of cancer in need thereof.

### SUMMARY OF THE INVENTION

This disclosure relates to methods of treating an individual at risk for incidence or recurrence of cancer in need thereof. The methods can include the steps of identifying an individual at risk for incidence or recurrence of cancer based on a risk stratification parameter, analyzing a biological sample from the individual at risk for incidence or recurrence of cancer using a multi-cancer detection (MCD) test to yield an MCD test result, and based on the MCD test result and optionally the risk stratification parameter, administering a neoantigen immunogenic composition to the individual at risk for incidence or recurrence of cancer in need thereof.

A risk stratification parameter can be any health parameter of the individual. The risk stratification parameter can include age, sex, family medical history, medications, germ-line mutations, somatic mutations, exposure to carcinogens, exposure to radiation, medical history, previous cancer diagnosis, or a combination of any of the foregoing.

A biological sample can be any sample from the individual at risk for incidence or recurrence of cancer. The biological sample can be an amniotic fluid sample, an ascitic fluid sample, a blood sample, a buccal sample, a cerebrospinal fluid sample, a fecal sample, a hair sample, a peritoneal fluid sample, a pleural effusion sample, a saliva sample, a semen sample, a synovial fluid sample, a tissue sample, a urine sample, or a combination of any of the foregoing.

In some aspects, the biological sample from the individual at risk for incidence or recurrence of cancer is a blood sample.

In some aspects, the biological sample from the individual at risk for incidence or recurrence of cancer is a tissue biopsy of a tumor or surgical tissue resection of a tumor.

The MCD test can analyze any biomarker of the individual. The MCD test can analyze nucleic acid methylation, nucleic acid mutation, nucleic acid fragmentation patterns, extracellular vesicle proteins, nucleic acid of circulating tumor cells, gene expression, circulating cancer antigens, or a combination of any of the foregoing.

The MCD test can include nucleic acid sequencing of a biological sample from an individual at risk for incidence or recurrence of cancer.

The MCD test can analyze any nucleic acid. For example, the MCD test can analyze cell free DNA (cfDNA), circulating tumor DNA (ctDNA), cell free RNA (cfRNA), circulating tumor RNA (ctRNA), or a combination of any of the foregoing.

The methods of this disclosure can include steps of sequencing and analyzing of a biological sample from the individual at risk for incidence or recurrence of cancer or a new biological sample from the individual at risk for incidence or recurrence of cancer to identify neoantigens to be included in the neoantigen immunogenic composition.

The methods of this disclosure can include further steps of determining the HLA type for an individual at risk for incidence or recurrence of cancer, scoring neoantigens of a biological sample from the individual at risk for incidence or recurrence of cancer, selecting neoantigens of the biological sample for inclusion in a neoantigen immunogenic composition, and/or generating the neoantigen immunogenic composition.

The methods described herein can include the conventional standard of care for the individual at risk for incidence or recurrence of cancer at the time of practicing the method is no treatment.

An individual at risk for incidence or recurrence of cancer can be a cancer survivor. The conventional standard of care for the individual at risk for incidence or recurrence of cancer at the time of practicing the method can be no treatment.

In some aspects, the individual at risk for incidence or recurrence of cancer cannot have been diagnosed with a cancer prior to the step of analyzing a biological sample.

The methods as disclosed herein, no tumor can be located in the individual at risk for incidence or recurrence of cancer.

In some aspects, the individual at risk for incidence or recurrence of cancer is diagnosed with cancer of unknown origin.

The conventional standard of care for the individual at risk for incidence or recurrence of cancer can exclude surgery, radiation, chemotherapy, immunotherapy, targeted therapy, hormone therapy, photodynamic therapy, cell therapy, or a combination of any of the foregoing.

The methods of this disclosure can include analyzing a second biological sample from the individual at risk for incidence or recurrence of cancer using a second multi-cancer detection (MCD) test during a course of treatment to determine efficacy of the neoantigen immunogenic composition.

In some aspects, a negative test result for the second MCD test indicates the neoantigen immunogenic composition is efficacious.

The methods described herein can include repeated steps at least once over a course of treatment for the individual at risk for incidence or recurrence of cancer. For example, steps of analyzing a biological sample from the individual at risk for incidence or recurrence of cancer using a multi-cancer detection (MCD) test to yield an MCD test result or, optionally, administering a neoantigen immunogenic composition to the individual at risk for incidence or recurrence of cancer in need thereof.

The individual at risk for incidence or recurrence of cancer can be diagnosed with chronic lymphocytic leukemia, ductal carcinoma in situ (DCIS), hormone receptor-positive breast cancer, non-Hodgkin's lymphoma, prostate cancer, or rectal cancer.

The individual at risk for incidence or recurrence of cancer can be diagnosed with stage 0 cancer, stage I cancer, or carcinoma in-situ.

The individual at risk for incidence or recurrence of cancer can be diagnosed with a pre-cancer or a benign cancer.

The methods disclosed herein, can further include sequencing nucleic acids of the biological sample to yield non-cancer sequencing results and cancer sequencing results, wherein the non-cancer sequencing results are subtracted from the cancer sequencing results to improve the signal to noise ratio for variant calling of neoantigens.

The methods of this disclosure further includes administering a co-therapy or procedure that is not the neoantigen immunogenic composition. The co-therapy or procedure can be a radiation treatment, a surgical procedure, a chemotherapeutic, an immunotherapy, a targeted therapy, a hormone therapy, a photodynamic therapy, a cell therapy, or a combination of any of the foregoing.

### BRIEF DESCRIPTION OF DRAWINGS

FIG. 1 is a flow chart that shows an embodiment of methods described herein for treating an individual at risk for incidence or recurrence of cancer. Individuals at risk for incidence or recurrence of cancer are identified by risk stratification parameters, such as exposure to carcinogens, family history, genetic susceptibility, aging, and status as a cancer survivor. A biological sample from an individual at risk for incidence or recurrence of cancer is analyzed by a multi-cancer detection (MCD) test. A positive MCD test result can result in no cancer found in the individual (e.g., no tumor is located in the individual), a confirmatory diagnosis of an early stage cancer, or a diagnosis of a cancer that does not warrant treatment by convention standard of care practices. In these cases, a neoantigen immunogenic composition (e.g., a cancer vaccine) is administered to the individual and further biological samples from the individual can be monitored for biomarkers of cancer (e.g., ctDNA, circulating cancer proteins). A subsequent negative test result for a biomarker (e.g., a negative MCD test result) can indicate the individual is no longer at risk for cancer or their cancer is in remission.

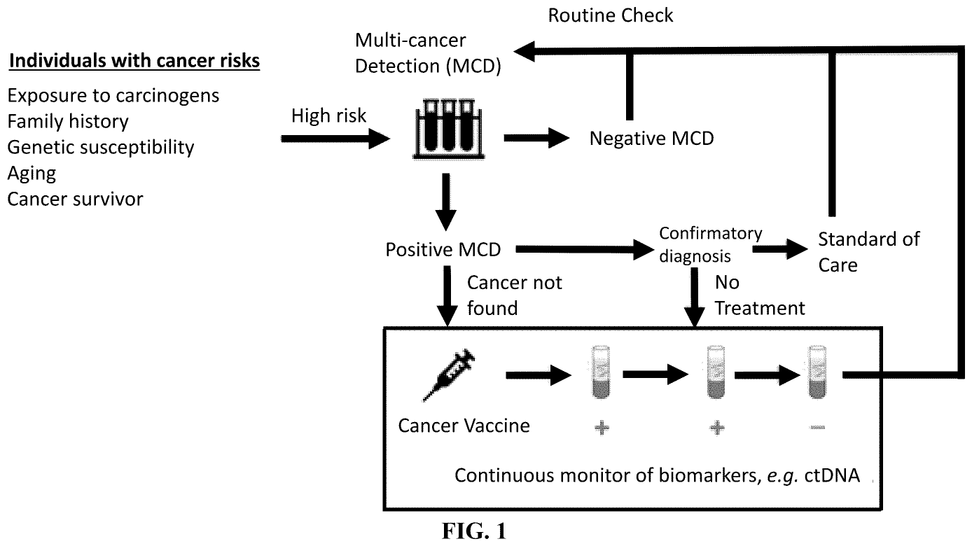

FIG. 2 is a flow chart showing an embodiment of methods described herein that include the step of generating a neoantigen immunogenic composition (e.g., a cancer vaccine). Following a positive MCD test result, nucleic acids from a biological sample of the individual at risk for incidence or recurrence of cancer can be sequenced and analyzed to predict neoantigens of the biological sample. The nucleic acids can be DNA (e.g., reused MCD test data, a targeted gene panel, whole genome sequencing (WGS), or whole exome sequencing (WES) results) or RNA (e.g., inferred from The Cancer Genome Atlas (TCGA) Program (e.g., see The Cancer Genome Atlas Network, et al. The Cancer Genome Atlas Pan-Cancer Analysis Project. Nature Genetics (2013) 45, 113-1120), predicted from methylation patterns, or sequenced from circulating RNA). The major histocompatibility complex (MHC, e.g., human leukocyte antigen (HLA)) can be typed for the individual and used to predict neoantigens. The neoantigens can be scored and ranked by predicted immunogenicity. Neoantigen peptides can be synthesized for inclusion in a neoantigen immunogenic composition (e.g., a cancer vaccine). Neoantigen peptides can be pooled in a neoantigen immunogenic composition for administration to the individual at risk for incidence or recurrence of cancer in need thereof.

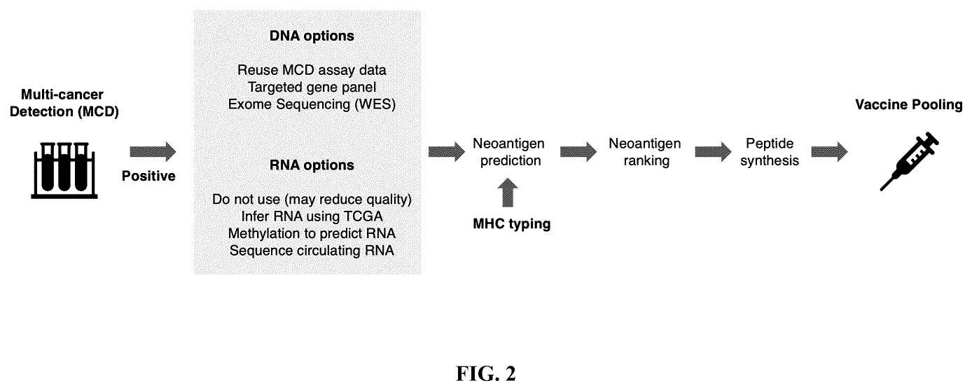

### DETAILED DESCRIPTION

The field of cancer treatment has advanced over the last two to three decades, with advancements in early detection of cancer (e.g., multi-cancer detection tests), new chemotherapeutic agents, and emerging immunotherapeutic treatments (e.g., chimeric T cell receptor therapies and check point inhibitor therapeutics). Despite recent innovations made by the research and medical community, a large subset of individuals including those with an early cancer diagnosis, elderly individuals, individuals with slowly growing cancers, cancer survivors, and individuals with cancer that is intractable to current therapies, are advised to wait for their cancer to advance in stage or become more malignant before treatment commences. This “watch and wait,” “active surveillance,” or “watchful waiting” approach is a conventional standard of care for many types of cancer, such as chronic lymphocytic leukemia, ductal carcinoma in situ (DCIS), hormone receptor-positive breast cancer, non-Hodgkin's lymphoma, prostate cancer, and rectal cancer. The same approach is also used for cancer survivors to monitor for cancer relapse (e.g., cancer recurrence) or other individuals at high risk of developing cancer but whom have not yet been diagnosed with cancer. The “watch and wait” or “watchful waiting” approach is intended to reduce the burden of side effects from oncology therapy, but often results in psychological trauma, such as anxiety or depression, that can be more distressful and have a greater negative impact on the quality of life than potential side effects of therapy or surgery (D. McCaughan, et al. Patient perspectives of ‘Watch and Wait’ for chronic haematological cancers: Findings from a qualitative study. Eur. J. Oncol. Nurs. (2023) 65, 102349, 1-9). To avoid the psychological trauma from the “watch and wait” or “active surveillance” conventional standard of care and/or symptoms associated with a slow-growing cancer, there is an urgent need for new methods of treating cancer that have few side effects and are capable of preventing and/or treating early stage cancer, undetectable cancers, undiagnosed cancers, relapsed cancer, or a combination of any of the foregoing. With such methods, clinicians could increase the efficacy of existing therapies and the chances of successful cancer treatment of an individual.

Described herein are methods of treating an individual at risk for incidence or recurrence of cancer in need thereof. The methods can have few or no side effects upon administration to the individual. The methods can include the steps of identifying an individual at risk for incidence or recurrence of cancer based on a risk stratification parameter; analyzing a biological sample from the individual at risk for incidence or recurrence of cancer using a multi-cancer detection (MCD) test to yield a MCD test result; and based on the MCD test result (e.g., a positive MCD test result) and optionally the risk stratification parameter, administering a neoantigen immunogenic composition to the individual at risk for incidence or recurrence of cancer in need thereof. The methods can be useful in treating individuals for whom the conventional standard of care is no treatment (e.g., “watch and wait” approach). It is believed, without wishing to be bound by any particular theory or mode of action, that methods of identification and treatment of individuals at high risk for developing cancer or at high risk for cancer recurrence with a neoantigen immunogenic composition (e.g., a personalized cancer vaccine) are efficacious in reducing the risk of developing cancer, treating cancer, and/or reducing the risk of cancer relapse. Advantageously, the methods described herein can be effective in treating individuals for whom the conventional standard of care is no treatment (e.g., “watch and wait” standard of care) because of the few or no side effects associated with administration of a neoantigen immunogenic composition. It is further believed that, by intervening earlier than advised by conventional standard of care treatments, methods described herein can reduce the risk of aggressive cancer incidence, increase the chance of cancer ablation, and are more efficacious compared to conventional standard of care treatments. Methods described herein can reduce the psychological symptoms (e.g., anxiety, depression) and/or the symptoms associated with slow-growing cancer that occur in individuals who would otherwise receive no treatment (e.g., individuals subject to a “watch and wait” standard of care).

Methods of treating an individual at risk for incidence or recurrence of cancer in need thereof described herein can include the step of identifying an individual at risk for incidence or recurrence of cancer based on a risk stratification parameter. As described herein, any risk stratification parameter can be used to identify an individual at risk for incidence or recurrence of cancer, including but not limited to age, sex, family medical history, medications, germ-line mutations, somatic mutations, past exposure to carcinogens, past exposure to radiation, medical history, previous cancer diagnosis, or a combination of any of the foregoing. Any number of risk parameters can be used to identify an individual at risk for incidence or recurrence of cancer.

Methods described herein can include the step of analyzing a biological sample from the individual at risk for incidence or recurrence of cancer using a multi-cancer detection (MCD) test to yield an MCD test result. The biological sample can be any type of biological sample including but not limited to an amniotic fluid sample, an ascitic fluid sample, a blood sample, a buccal sample, a cerebrospinal fluid sample, a fecal sample, a hair sample, a peritoneal fluid sample, a pleural effusion sample, a saliva sample, a semen sample, a synovial fluid sample, a tissue sample, a urine sample, or a combination of any of the foregoing. Any number of biological samples from an individual can be analyzed. As described herein, the MCD test can be any type of MCD test. The MCD test can analyze any parameter of a biological sample, including nucleic acid methylation, nucleic acid mutation, nucleic acid fragmentation patterns, extracellular vesicle proteins, nucleic acid of circulating tumor cells, gene expression, circulating cancer antigens, or a combination of any of the foregoing. In some embodiments, the MCD test can analyze a nucleic acid that is cell free DNA (cfDNA), circulating tumor DNA (ctDNA), cell free RNA (cfRNA), circulating tumor RNA (ctRNA), or a combination of any of the foregoing.

Methods described herein can include the step of, based on an MCD test result (e.g., a positive MCD test result) and optionally a risk stratification parameter, administering a neoantigen immunogenic composition to the individual at risk for incidence or recurrence of cancer in need thereof. For example, the MCD test can sequence cfDNA, the MCD test result can identify sequence variants (e.g., somatic mutations, neoantigens), and the risk stratification parameter can be the age of an individual that is at least 72 years, resulting in administering a neoantigen immunogenic composition to the individual in need thereof. The step of administering a neoantigen immunogenic composition can further include administering a co-therapy or procedure that (i) is not the neoantigen immunogenic composition and (ii) is a radiation treatment, a surgical procedure, a chemotherapeutic, an immunotherapy, a targeted therapy, a hormone therapy, a photodynamic therapy, a cell therapy, or a combination of any of the foregoing.

Methods described herein can include the step of sequencing and analyzing of the biological sample from an individual at risk for incidence or recurrence of cancer or a new biological sample from the individual at risk for incidence or recurrence of cancer to identify neoantigens to be included in a neoantigen immunogenic composition. As described herein, any method can be used to identify, score, and/or select neoantigens for neoantigen immunogenic composition. Methods can further include the steps of determining the HLA type for the individual at risk for incidence or recurrence of cancer; scoring neoantigens of the biological sample from the individual at risk for incidence or recurrence of cancer; selecting neoantigens of the biological sample for inclusion in a neoantigen immunogenic composition, and/or generating the neoantigen immunogenic composition.

Methods described herein can include the step of analyzing a second biological sample from the individual at risk for incidence or recurrence of cancer using a second multi-cancer detection (MCD) (e.g., during the course of treatment) test to determine efficacy of the neoantigen immunogenic composition. In such embodiments, a negative test result for the second MCD test can indicate the neoantigen immunogenic composition is efficacious.

Methods described herein can include the step of sequencing nucleic acids of the biological sample to yield non-cancer sequencing results and cancer sequencing results. The non-cancer sequencing results can be subtracted from the cancer sequencing results to improve the signal to noise ratio for variant calling of neoantigens.

In some embodiments, methods contain the steps of identifying the individual at risk for incidence or recurrence of cancer based on a risk stratification parameter, analyzing a biological sample from the individual at risk for incidence or recurrence of cancer using a multi-cancer detection (MCD) test to yield an MCD test result, and, based on the MCD test result, and optionally the risk stratification parameter, administering a neoantigen immunogenic composition to the individual at risk for incidence or recurrence of cancer in need thereof.

Methods described herein or any steps of the methods described herein can be repeated over the duration of a treatment or following a treatment (e.g., following an administration of a neoantigen immunogenic composition, following administration of a neoantigen cancer vaccine) of an individual. The steps of analyzing a biological sample from the individual at risk for incidence or recurrence of cancer using a multi-cancer detection (MCD) test to yield an MCD test result and, based on the MCD test result and optionally the risk stratification parameter, administering a neoantigen immunogenic composition to the individual at risk for incidence or recurrence of cancer in need thereof.

The method can further include generating the neoantigen immunogenic composition comprising the steps of sequencing and analyzing of the biological sample from the individual at risk for incidence or recurrence of cancer or a new biological sample from the individual at risk for incidence or recurrence of cancer to identify neoantigens to be included in the neoantigen immunogenic composition, determining the HLA type for the individual at risk for incidence or recurrence of cancer, scoring neoantigens of the biological sample from the individual at risk for incidence or recurrence of cancer, selecting neoantigens of the biological sample for inclusion in the neoantigen immunogenic composition, generating the neoantigen immunogenic composition, analyzing a second biological sample from the individual at risk for incidence or recurrence of cancer using a second multi-cancer detection (MCD) (e.g., during the course of treatment) test to determine efficacy of the neoantigen immunogenic composition, sequencing nucleic acids of the biological sample to yield non-cancer sequencing results and cancer sequencing results, wherein the non-cancer sequencing results are subtracted from the cancer sequencing results to improve the signal to noise ratio for variant calling of neoantigens, and/or combinations of any of the foregoing steps of a method can be repeated over the course of treatment for an individual at risk for incidence or recurrence of cancer.

The number of repetitions of a method or steps of the method over the course of treatment can be about 1, about 2, about 3, about 4, about 5, about 6, about 7, about 8, about 9, about 10, about 11, about 12, about 13, about 14, or about 15. The number of repetitions of a method or steps of the method can be at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20. The number of repetitions of a method or steps of the method can be between about 1 to about 20, about 1 to about 19, about 1 to about 18, about 1 to about 17, about 1 to about 16, about 1 to about 15, about 1 to about 14, about 1 to about 13, about 1 to about 12, about 1 to about 11, about 1 to about 10, about 1 to about 9, about 1 to about 8, about 1 to about 7, about 1 to about 6, about 1 to about 5, about 1 to about 4, about 1 to about 3, about 1 to about 2, about 2 to about 20, about 2 to about 19, about 2 to about 18, about 2 to about 17, about 2 to about 16, about 2 to about 15, about 2 to about 14, about 2 to about 13, about 2 to about 12, about 2 to about 11, about 2 to about 10, about 2 to about 9, about 2 to about 8, about 2 to about 7, about 2 to about 6, about 2 to about 5, about 2 to about 4, about 2 to about 3, about 3 to about 20, about 3 to about 19, about 3 to about 18, about 3 to about 17, about 3 to about 16, about 3 to about 15, about 3 to about 14, about 3 to about 13, about 3 to about 12, about 3 to about 11, about 3 to about 10, about 3 to about 9, about 3 to about 8, about 3 to about 7, about 3 to about 6, about 3 to about 5, or about 3 to about 4. In certain embodiments, the step of analyzing a biological sample from an individual at risk for incidence or recurrence of cancer using an MCD test to yield an MCD test result and optionally the step of based on the MCD test result and optionally the risk stratification parameter administering a neoantigen immunogenic composition to the individual at risk for incidence or recurrence of cancer in need thereof are repeated at least once over a course of treatment for the individual at risk for incidence or recurrence of cancer. The number of steps of the method can be skipped, removed or altered. Repetition of the method may vary in number of steps of the method.

The duration of time between repetitions of the method or steps of the method can be about 1 day, about 2 days, about 3 days, about 4 days, about 5 days, about 6 days, about 7 days, about 8 days, about 9 days, about 10 days, about 12 days, about 14 days, about 16 days, about 18 days, about 20 days, about 22 days, about 24 days, about 26 days, about 28 days, about 30 days, about 1 month, about 2 months, about 3 months, about 4 months, about 5 months, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months about 22 months, about 23 months, or about 2 years. The duration of time between repetitions of the method or steps of the method can be at least about 1 day, at least about 2 days, at least about 3 days, at least about 4 days, at least about 5 days, at least about 6 days, at least about 7 days, at least about 8 days, at least about 9 days, at least about 10 days, at least about 12 days, at least about 14 days, at least about 16 days, at least about 18 days, at least about 20 days, at least about 24 days, at least about 28 days, at least about 1 month, at least about 2 months, at least about 4 months, at least about 6 months, at least about 8 months, at least about 10 months, at least about 1 year, at least about 1.5 years, or at least about 2 years.

### Definitions

All publications and patents cited in this disclosure are incorporated by reference in their entirety. To the extent the material incorporated by reference contradicts or is inconsistent with this specification, the specification will supersede any such material. The citation of any references herein is not an admission that such references are prior art to the present disclosure. Various terms relating to aspects of the description are used throughout the specification and claims. Such terms are to be given their ordinary meaning in the art unless otherwise indicated. Other specifically defined terms are to be construed in a manner consistent with the definitions provided herein.

When a range of values is expressed, it includes embodiments using any particular value within the range. Further, reference to values stated in ranges includes each and every value within that range. All ranges are inclusive of their endpoints and combinable. When values are expressed as approximations, by use of the antecedent “about,” it will be understood that the particular value forms another embodiment. Reference to a particular numerical value includes at least that particular value, unless the context clearly dictates otherwise. The use of “or” will mean “and/or” unless the specific context of its use dictates otherwise.

Unless otherwise indicated, the terms “at least,” “less than,” “about,” and “at most,” or similar terms preceding a series of elements or a range are to be understood to refer to every element in the series or range. Those skilled in the art will recognize, or be able to ascertain using no more than routine experimentation, many equivalents to the specific embodiments of the invention described herein. Such equivalents are intended to be encompassed by the following claims.

As used herein, the singular forms “a,” “an,” and “the” include plural forms unless the context clearly indicates otherwise. The terms “include,” “such as,” and the like are intended to convey inclusion without limitation, unless otherwise specifically indicated.

As used herein, the term “cancer” refers to the physiological condition in subjects in which a population of cells is characterized by uncontrolled proliferation, immortality, metastatic potential, rapid growth and proliferation rate, and/or certain morphological features. Often cancers can be in the form of a tumor or mass, but may exist alone within the subject, or may circulate in the blood stream as independent cells, such as lymphoma cells. The term cancer includes all types of cancers and metastases, including hematological malignancy, solid tumors, sarcomas, carcinomas, and other solid and non-solid tumors. Examples of cancers include, but are not limited to, carcinoma, lymphoma, blastoma, sarcoma, and leukemia. More particular examples of such cancers include squamous cell cancer, small cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer (e.g., triple negative breast cancer, hormone receptor positive breast cancer), osteosarcoma, melanoma, colon cancer, colorectal cancer, endometrial (e.g., serous) or uterine cancer, salivary gland carcinoma, kidney cancer, liver cancer, prostate cancer, vulvar cancer, thyroid cancer, hepatic carcinoma, and various types of head and neck cancers. Triple negative breast cancer refers to breast cancer that is negative for expression of the genes for estrogen receptor (ER), progesterone receptor (PR), and Her2/neu. Hormone receptor positive (e.g., hormone-positive) breast cancer refers to breast cancer that is positive for at least one of the following: ER or PR, and negative for Her2/neu (HER2).

As used herein, the term “individual at risk for incidence or recurrence of cancer” refers to an individual with elevated an elevated risk of being diagnosed with cancer, developing cancer, and/or having a relapsed cancer (e.g., cancer recurrence). As described herein, an individual can be identified as an individual at risk for incidence or recurrence of cancer based on any risk stratification parameter, such as age, sex, family medical history, medications, germ-line mutations, somatic mutations, past exposure to carcinogens, past exposure to radiation, medical history, previous cancer diagnosis, or a combination of any of the foregoing. In some embodiments, the individual at risk for incidence or recurrence of cancer is a cancer survivor.

Any terms not directly defined herein shall be understood to have the meaning commonly associated with them as understood within the art of the invention. Any terms not directly defined herein shall be understood to have the meanings commonly associated with them as understood within the art of the invention. Certain terms are discussed herein to provide additional guidance to the practitioner in describing the compositions, devices, methods, and the like of aspects of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

### Risk Stratification Parameters

Methods described herein can identify an individual at risk for incidence or recurrence of cancer based on a risk stratification parameter. The risk stratification parameter can be any information from an individual, including but not limited to age, sex, family medical history, medications, germ-line mutations, somatic mutations, exposure to carcinogens, exposure to radiation, medical history, previous cancer diagnosis, or a combination of any of the foregoing. For example, methods of this disclosure can include the step of identifying an individual at risk for incidence or recurrence of cancer based on a risk stratification parameter that is age (e.g., age of the individual, over 67 years of age).

The risk stratification parameter can be an individual's age. An individual's age can be used as a metric to calculate the risk of cancer in the individual (e.g., age can be used as a threshold for determining risk of cancer or identifying an individual at risk for incidence or recurrence of cancer). The age of the individual can be directly related to the risk of cancer in the individual (e.g., an individual aged 50 years has higher risk than an individual aged 19 years). The age threshold for determining an individual at risk for incidence or recurrence of cancer can be any age, including but not limited to an age of at least 20 years, at least 25 years, at least 30 years, at least 35 years, at least 40 years, at least 45 years, at least 50 years, at least 55 years, at least 60 years, at least 65 years, at least 66 years, at least 67 years, at least 68 years, at least 69 years, at least 70 years, at least 71 years, at least 72 years, at least 73 years, at least 74 years, at least 75 years, at least 76 years, at least 77 years, at least 78 years, at least 79 years, at least 80 years, at least 81 years, at least 82 years, at least 83 years, at least 84 years, at least 85 years, at least 86 years, at least 87 years, at least 88 years, at least 89 years, at least 90 years, at least 91 years, at least 92 years, at least 93 years, at least 94 years, at least 95 years, at least 96 years, at least 97 years, at least 98 years, at least 99 years, at least 100 years, at least 101 years, at least 102 years, at least 103 years, at least 104 years, or at least 105 years. For example, methods of this disclosure can be used to identify individuals at risk for incidence or recurrence of a cancer associated with somatic mutations in older individual populations (e.g., prostate cancer, colon cancer). The age of the individual can be inversely related to the risk of cancer in the individual (e.g., an individual aged 3 years has a higher risk than an individual aged 50 years). The age threshold for determining an individual at risk for incidence or recurrence of cancer can be an age of at most 45 years, at most 40 years, at most 35 years, at most 30 years, at most 25 years, at most 24 years, at most 23 years, at most 22 years, at most 21 years, at most 20 years, at most 19 years, at most 18 years, at most 17 years, at most 16 years, at most 15 years, at most 14 years, at most 13 years, at most 12 years, at most 11 years, at most 10 years, at most 9 years, at most 8 years, at most 7 years, at most 6 years, at most 5 years, at most 4.5 years, at most 4 years, at most 3.5 years, at most 3 years, at most 2.5 years, at most 2 years, at most 1.5 years, at most 1 year, or at most 6 months. For example, methods of this disclosure can be used to identify individuals at risk for incidence or recurrence of cancer for a juvenile or childhood cancer (e.g., a childhood leukemia or Wilms tumor). In such an example, the age of the individual is inversely correlated with risk of childhood cancer and the age threshold could be at most 13 years.

The risk stratification parameter can be an individual's sex. For example, sex of an individual can be used to identify individuals that are at high risk for a cancer associated with, occurs at a higher incidence in, or exclusive to biological males (e.g., prostate cancer, penile cancer, testicular cancer). For example, sex of an individual can be used to identify individuals that are at high risk for a cancer associated with, occurs at a higher incidence in, or exclusive to biological females (e.g., ovarian cancer, cervical cancer, uterine cancer, breast cancer).

The risk stratification parameter can be family medical history (e.g., family medical history of an individual's relatives). Family medical history includes but is not limited to family history of death from cancer, cancer incidence, cancer survivors, ongoing treatment for cancer, cancer metastasis, drug-resistant cancer, recurrence of cancer, and a combination of any of the foregoing. For example, the risk stratification parameter can account for lung cancer incidence in family members of an individual to identify if the individual is at risk for incidence or recurrence of cancer.

The risk stratification parameter can be medication (e.g., medication history of an individual, current medications of an individual). Any factor of medication can be considered including, but not limited to current medications, past medications, length of treatment with a medication, dosage of treatment with a medication, manufacturer, lot number, batch number, frequency of dosage, route of administration, age of administration, expiration date of medication, or combinations of any of the foregoing. Medications identified as correlated with cancer incidence can be considered in a risk stratification medication. For example, use of a hormone therapy, birth control, proton pump inhibitor (PPI), angiotensin blockers (e.g., irbesartan, losartan, telmisartan, valsartan), diethylstilbestrol, metformin, immunosuppressants (e.g., immunosuppressive treatments for organ transplant), azathioprine, or other medications associated with increased cancer risk can be a risk stratification parameter in methods described herein.

The risk stratification parameter can be any genetic mutation (e.g., any germ-line mutation, somatic mutation) of an individual. The genetic mutation can be in any gene including, but is not limited to APC, ATM, AXIN2, BAP1, BMPR1A, BRCA1, BRCA2, BRIP1, CDC73, CDH1, CDK4, CDKN2A, CHEK2, DICER1, EGFR, EPCAM, FH, FLCN, GREM1, HERC2, HOXB13, KIT, MEN1, MET, MLH1, MSH2, MSH6, MUTYH, NF1, NF2, PALB2, PDGFRA, PMS2, POLD1, POLE, PTCH, PTEN, RAD51C, RAD51D, RET, SHDA, SDHB, SDHC, SDHD, SDHAF2, SMAD4, STK11, SUFU, TP53, VHL, and combinations of any of the foregoing. For example, the presence of homozygous BRCA1 mutation in an individual can be a risk stratification parameter used in methods to identify an individual at risk for incidence or recurrence of cancer. The risk stratification parameter can be a somatic mutation identified in any gene of an individual (e.g., identified by multi-cancer detection test, identified by sequencing of nucleic acids from an individual, identified by nucleic acid sequencing of a biological sample from the individual) including, but not limited to ANK1, ARID1A, ATM, BCAS3, BMPR1B, CASC8, CCND1, CCNE1, CDKN2A-DT, CDKN2A, CDKN2B, CSMD1, EGFR, ELAVL2, EPHA3, ERBB2, FAT1, GSDMA, KDM5A, KMT2C, KMT2D, KRAS, LPAR6, LRP1B, MGA, miR-1204, MITF, MTAP, MYC, NF1, NFE2L2, ODZ3, ORAOV1, PIK3CA, PLXNB2, PTEN, RARG, RB1, RNA5SP251, SCG3, SMAD4, TAL1, TERT, TP53, TSHZ3, WWOX, and combinations of any of the foregoing. For example, nucleic acid sequencing of blood from an individual can identify an individual at risk for incidence or recurrence of cancer by the presence of a mutation in the MYC gene in cell free DNA (cfDNA).

The risk stratification parameter can be exposure of the individual to carcinogens. The carcinogen can be any carcinogen including but not limited to aflatoxin, alcohol, 4-aminobiphenyl, arsenic (e.g., inorganic arsenic compounds), aristolochic acids, asbestos, benzene, benzidine, beryllium (e.g., beryllium compounds), bis(chloromethyl) ether, 1,3-butadiene, 1,4-butanediol dimethanesulfonate, cadmium (e.g., cadmium compounds), chloromethyl methyl ether, chlorambucil, chromium hexavalent compounds, coal tars, coal tar pitches, coke-oven emissions, cyclophosphamide, cyclosporin A, diethylstilbestrol, dyes metabolized to benzidine, erionite, estrogens, ethylene oxide, formaldehyde, melphalan, methoxsalen with ultraviolet A therapy, mineral oils, mustard gas, 2-naphthylamine, nickel compounds, nicotine, nitrosourea agents, silica (e.g., crystalline silica), soots, strong inorganic acid mists containing sulfuric acid, tamoxifen, 2,3,7,8-tetrachlorodibenzo-p-dioxin, thiotepa, thorium dioxide, tobacco (e.g., smokeless tobacco), tobacco smoke, o-toluidine, trichloroethylene, vinyl halide (e.g., vinyl chloride), wood dust, and combinations of any of the foregoing. Exposure to carcinogens can be from any source including but not limited to tobacco use (e.g., smoking, chew tobacco), dietary exposure (e.g., aflatoxin contaminated peanuts, processed food, cured meats), contaminated water, air pollution, occupational exposure to carcinogens, and combinations of any of the foregoing. For example, occupational exposure to secondhand smoke can be a risk stratification parameter used to identify individuals at risk for incidence or recurrence of cancer in methods described herein.

The risk stratification parameter can be exposure of the individual to radiation. The radiation can be any type of radiation associated with cancer risk including but not limited to ionizing radiation (e.g., gamma radiation, X-ray, alpha decay radiation, beta particle decay radiation, neutron radiation), ultraviolet radiation (e.g., UVA radiation, UVB radiation), and combinations of any of the foregoing.

The risk stratification parameter can be an individual's medical history. The risk stratification parameter can be any aspect of medical history, including but not limited to status as a cancer survivor, infection history, status as an organ recipient, known diseases or disorders of the individual (e.g., type 2 diabetes, cardiovascular disease, obesity, pulmonary disease, inflammatory bowel disease, arthritis), biometrics (e.g., blood pressure, cholesterol level, heart rate, body mass index, percent body fat) of individual, and combinations of any of the foregoing. An infection history can be any infection, including but not limited to infections known to cause, be associated with, or contribute to cancer, such as Bacteroides fragilis, Chlamydia pneumoniae, Epstein-Barr virus (EBV), a human immunodeficiency virus (e.g., HIV-1, HIV-2), a human papillomavirus (HPV), a human T-lymphotropic virus (e.g., HTLV-1, HTLV-2), Kaposi's sarcoma-associated herpesvirus (KSHV), Merkel cell polyomavirus (MCPyV), a Helicobacter sp. (e.g., H. pylori, H. salomonis, H. suis, H. heilmannii sensu lato, H. bizzozeronii), a hepatitis virus (e.g., Hepatitis B virus (HBV), Hepatitis C virus (HCV)), Opisthorchiasis sp. (e.g., O. viverrini), Schistosoma sp. (e.g., S. haematobium, S. japonicum), E. coli, Mycobacterium sp. (e.g., M. tuberculosis), Plasmondium sp. (e.g., P. falciparum, P. vivax, P. ovale, P. malariae), and the like. The risk stratification parameter can include past cancer diagnosis (e.g., stage of cancer, grade of cancer, origin of cancer, type of cancer) of the individual. The previous cancer diagnosis can be any stage, grade, origin, or type of cancer, including but not limited to those described herein. The previous cancer diagnosis can be a tumor at any bodily site, such as the bodily sites described herein. The previous cancer can be in remission, currently undergoing treatment, under “active surveillance,” undetectable, cured, or a combination of any of the foregoing.

Methods described herein can identify individuals at risk for incidence or recurrence of cancer based by any number of different risk stratification parameters. The number of risk stratification parameters can be about 1, about 2, about 3, about 4, about 5, about 6, about 7, about 8, about 9, about 10, about 11, about 12, about 13, about 14, about 15, about 16, about 17, about 18, or about 20. The number of risk stratification parameters can be at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20. The number of risk stratification parameters can be at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, or at most 20. The number of risk stratification parameters can be between about 1 to about 20, about 2 to about 20, about 3 to about 20, about 4 to about 20, about 5 to about 20, about 6 to about 20, about 7 to about 20, about 8 to about 20, about 9 to about 20, about 10 to about 20, about 11 to about 20, about 12 to about 20, about 13 to about 20, about 14 to about 20, about 15 to about 20, about 16 to about 20, about 17 to about 20, about 18 to about 20, about 19 to about 20, about 1 to about 19, about 1 to about 18, about 1 to about 17, about 1 to about 16, about 1 to about 15, about 1 to about 14, about 1 to about 13, about 1 to about 12, about 1 to about 11, about 1 to about 10, about 1 to about 9, about 1 to about 8, about 1 to about 7, about 1 to about 6, about 1 to about 5, about 1 to about 4, about 1 to about 3, about 1 to about 2, about 2 to about 10, about 2 to about 5, about 3 to about 10, about 3 to about 5, about 4 to about 10, or about 4 to about 5. For example, methods of this disclosure can include the step of identifying an individual at risk for incidence or recurrence of cancer based on three risk stratification parameters: family medical history, medical history of the individual, and germ-line mutations.

### Biological Samples

Biological samples used in methods of this disclosure can be obtained using any methodology and contain tissue or fluid from any bodily site, organ, or of any tissue type. The biological sample from the individual at risk for incidence or recurrence of cancer can be an amniotic fluid sample, an ascitic fluid sample, a blood sample, a buccal sample, a cerebrospinal fluid sample, a fecal sample, a hair sample, a peritoneal fluid sample, a pleural effusion sample, a saliva sample, a semen sample, a synovial fluid sample, a tissue sample, a urine sample, or a combination of any of the foregoing. A biological sample (e.g., a tissue sample) can be a biopsy sample (e.g., a tissue biopsy sample). A biopsy sample can be any type of biopsy including, but not limited to, an excisional biopsy, a liquid biopsy, an incisional biopsy, a needle biopsy, a punch biopsy, or a shave biopsy. A needle biopsy can be any type of needle biopsy including but not limited to, a core needle biopsy, a fine-needle aspiration biopsy, or a combination thereof. A needle biopsy can have any needle gauge including, but not limited to, 9, 10, 11, 12, 13, 14, 15, 16, 17, 18, 19, 20, 21, 22, 23, 24, 25, 26, 27, 28, 29, or 30. The needle biopsy gauge can range from 9 to 18, 10 to 17, 11 to 16, 12 to 15, 13 to 14, 20 to 30, 21 to 30, 22 to 30, 23 to 30, 9 to 30, 9 to 29, 9 to 28, 9 to 27, 9 to 26, 9 to 25, 9 to 24, 9 to 23, 9 to 22, 9 to 21, 9 to 20, 9 to 19, 9 to 17, 9 to 16, 9 to 15, 9 to 14, 9 to 13, 9 to 12, 9 to 11, 9 to 10, 10 to 30, 11 to 30, 12 to 30, 13 to 30, 14 to 30, 15 to 30, 16 to 30, 17 to 30, 18 to 30, or 19 to 30.

A biological sample can be a liquid biopsy. The liquid biopsy can be any biological fluid including, but not limited to an amniotic fluid sample, a blood sample, a buccal sample, a cerebrospinal fluid sample, a fecal sample, a peritoneal fluid sample, a pleural effusion sample (e.g., a transudative pleural effusion sample, an exudative effusion sample), a saliva sample, a semen sample, a synovial fluid sample, a urine sample, or a combination thereof. In contrast to a tissue biopsy, which comprises primarily solid biological tissue and is collected through a surgical procedure, a liquid biopsy can be collected from a vein or artery of the individual. The liquid biopsy can contain blood, circulating tumor cells, circulating tumor material (e.g., circulating tumor DNA (ctDNA), circulating tumor extracellular vesicles, circulating tumor proteins, circulating tumor RNA), plasma, serum, healthy blood cells, or a combination thereof. A liquid biopsy can be taken from an individual suffering from any type of disorder or condition including, but not limited to, an infection and a cancer (e.g., a hematological cancer, a solid tumor). In some embodiments, the biological sample from the individual at risk for incidence or recurrence of cancer is a blood sample.

Biological samples can include two or more different biological sample types (e.g., two or more different biopsy types). For example, biological samples used in methods described herein can include three biopsy samples: two liquid biopsy samples and one tissue needle biopsy sample. Biological samples can include two or more of the same biopsy types. For example, the biological sample can include three tissue biopsy samples that are core needle biopsies. For example, the biological sample can include three liquid biopsy samples that are blood samples. The number of different biological sample types can be 1, 2, 3, 4, 5, 6, 7, 8, 9, 10, 11, 12, 13, 14, 15, 16, 17, 18, 19, or 20. The number of biological sample types can be at least 2, at least 3, at least 4, at least 5, at least 6, at least 7, at least 8, at least 9, or at least 10.

A biological sample (e.g., a tissue sample) can be any surgically removed tissue from an individual including, but not limited to, an adenectomy sample, adenoidectomy, adrenalectomy sample, apicoectomy sample, appendectomy sample, auriculectomy sample, bullectomy sample, bunionectomy sample, cecectomy sample, cervicectomy sample, cholecystectomy sample, colectomy sample, craniectomy sample, cystectomy sample, corpectomy sample, discectomy sample, diverticulectomy sample, duodenectomy sample, esophagectomy sample, extrapleural pneumonectomy sample, frenectomy sample, fundectomy sample, ganglionectomy sample, gastrectomy sample, gingivectomy sample, glossectomy sample, gonadectomy sample, hemicolectomy sample, hemilaminectomy sample, hemipelvectomy sample, hemispherectomy sample, hemorrhoidectomy sample, hepatectomy sample, hypophysectomy sample, hysterectomy sample, iridectomy sample, jejunectomy sample, keratectomy sample, laminectomy sample, laryngectomy sample, lumpectomy sample, lymphadenectomy sample, mastectomy sample, mastoidectomy sample, myocardiectomy sample, myomectomy sample, necrosectomy sample, nephrectomy sample, neurectomy sample, oophorectomy sample, orchiectomy sample, ostectomy sample, pancreatectomy sample, pancreaticoduodenectomy sample, panniculectomy sample, parathyroidectomy sample, pericardiectomy sample, pinealectomy sample, pneumonectomy sample, proctocolectomy sample, prostatectomy sample, pulpectomy sample, quadrantectomy sample, rhinectomy sample, salpingectomy sample, salpingo-oophorectomy sample, septectomy sample, splenectomy sample, stapedectomy sample, sympathectomy sample, thymectomy sample, thyroidectomy sample, tonsillectomy sample, trabeculectomy sample, tumor sample (e.g., a tumorectomy, a surgically excised tumor), turbinectomy sample, tubectomy sample, uterectomy sample, uvulectomy sample, vitrectomy sample, or combinations thereof. The biological sample can be from healthy tissue (e.g., non-cancerous tissue). The biological sample can be from cancerous tissue (e.g., a tumor). In some embodiments, the biological sample is a tissue sample from a tissue biopsy of a tumor or surgical tissue resection of a tumor.

A biological sample (e.g., a tissue biopsy, a surgically removed tissue, a liquid biopsy) can be from any anatomical position. Anatomical positions include, but are not limited to, abdomen, ankle, arm, back, brachium, breast, buttocks, calf, chest, ear, elbow, eye, face, finger, foot, forearm, genitalia, hand, head, hip, knee, leg, mouth, neck, nose, scalp, shin, shoulder, thigh, toe, waist, and wrist. A biological sample can be from any organ including, but not limited to, adrenal glands, appendix, anus, artery, bladder, bone marrow, brain, bronchi, bronchioles, capillary, cervix, colon, ear, epididymis, esophagus, eye, fallopian tube, gallbladder, gut-associated lymphoid tissue, heart, interstitium, joint, kidney, large intestine, larynx, ligament, liver, lung, lymph node, mammary gland, mesentery, mouth, muscle, nasal cavity, nerve, olfactory epithelium, ovary, pancreas, pharynx, pineal gland, pituitary gland, placenta, prostate, rectum, salivary gland, skeleton, skin, small intestine, spinal cord, spleen, stomach, subcutaneous tissue, tendon, testicle, thymus, thyroid gland, tongue, trachea, ureter, urethra, uterus, vas deferens, ventricular system, and vein. The biological sample (e.g., tissue biopsy sample, a surgically removed tissue) can contain any bodily tissue including, but not limited to, connective tissue, epithelial tissue, muscular tissue, nervous tissue, or combinations thereof.

Methods described herein can be practiced on two or more biological samples from an individual. The number of biological samples can be 2, 3, 4, 5, 6, 7, 8, 9, 10, 11, 12, 13, 14, 15, 16, 17, 18, 19, or 20. The number of biological samples can be at least about 2, at least about 3, at least about 4, at least about 5, at least about 6, at least about 7, at least about 8, at least about 9, at least about 10, at least about 11, at least about 12, at least about 13, at least about 14, or at least about 15. Biological samples from an individual can be from the same region of the body of an individual. For example, three tissue samples could be from the left lung of an individual. Biological samples can be from different regions of the body of an individual. For example, a first tissue sample could be from the pancreas, and a second tissue sample could be from the bile duct of an individual. The number of biological samples from different regions of the body of an individual can be at least two, at least three, at least four, at least five, at least six, at least seven, at least eight, at least nine, or at least ten. Biological samples can be from the same region of a tumor of an individual. Biological samples can be from different regions of a tumor of an individual. For example, two tissue samples can include a first tissue sample that is collected from the posterior of an adenocarcinoma tumor and the second tissue sample is collected from the anterior of the same adenocarcinoma tumor. The number of biological samples from different regions of a tumor of an individual can be at least two, at least three, at least four, at least five, at least six, at least seven, at least eight, at least nine, or at least ten. Biological samples from an individual can be from a primary tumor of the individual. Biological samples from an individual can be from a secondary (e.g., metastatic tumor) of the individual. Two or more biological samples (e.g., tissue samples) can be from a different type of tumor including a primary tumor and a secondary tumor. For example, two tissue samples (e.g., a first tissue sample and a second tissue sample) could be collected and include a first tissue sample from a primary tumor from the liver and a second tissue sample could be from a metastatic tumor in the brain of an individual. In some embodiments, at least two of the biological samples are from different regions of the body of an individual. In some embodiments, at least two of the biological samples are from different regions of a tumor of an individual. In some embodiments, at least two of the biological samples are from different regions of a tumor of an individual.

The biological sample (e.g., tissue sample) can be an archival sample, such as an archival fluid or an archival tissue sample. Archival samples (e.g., archival fluid samples, archival tissue samples, archival bone marrow smears) can be collected at any time point including, but not limited to prior to practicing methods or steps of methods described herein or during practicing methods or steps of methods described herein. Any type of archival fluid or tissue sample can be used, including but not limited to cancerous fluid samples, cancerous tissue samples, non-cancerous fluid samples, and/or non-cancerous tissue samples. The archival tissue sample can be an archival tumor sample (e.g., an archival tissue biopsy from a tumor or an archival surgically removed tissue from a tumor). The archival tissue sample can be collected at any time point in relation to practicing the methods and steps of methods described herein. The time interval separating the collection of the archival tissue and practicing methods or steps of methods described herein can be at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 1 month, at least 1.5 months, at least 2 months, at least 3 months, at least 4 months, at least 5 months, at least 6 months, at least 7 months, at least 8 months, at least 9 months, at least 10 months, at least 11 months, at least 12 months, at least 1 year, at least 14 months, at least 15 months, at least 16 months, at least 17 months, at least 18 months, at least 19 months, at least 20 months, at least 21 months, at least 22 months, at least 23 months, at least 2 years, at least 3 years, at least 4 years, at least 5 years, at least 6 years, at least 7 years, at least 8 years, at least 9 years, or at least 10 years.

The biological sample (e.g., the tissue biopsy sample, the surgically removed tissue, the liquid biopsy sample), can be collected and/or maintained at any temperature. For example, the biological sample can be collected and/or maintained at room temperature. For example, the biological sample can be collected and/or maintained frozen. The biological sample can be collected and/or maintained at about −78° C., about −76° C., about −74° C., about −72° C., about −70° C., about −68° C., about −66° C., about −64° C., about −62° C., about −60° C., about −58° C., about −56° C., about −54° C., about −52° C., about −50° C., about −48° C., about −46° C., about −44° C., about −42° C., about −40° C., about −38° C., about −36° C.,-about 34° C., about −32° C., about −30° C., about −28° C., about −26° C., about −24° C., about −22° C., about −20° C., about −18° C., about −16° C., about −14° C., about −12° C., about −10° C., about −8° C., about −6° C., about −4° C., about −2° C., about 0° C., about 2° C., about 4° C., about 6° C., about 8° C., about 10° C., about 12° C., about 14° C., about 16° C., about 18° C., about 20° C., about 22° C., about 24° C., about 26° C., about 28° C., about 30° C., about 32° C., or about 34° C. The biological sample can be collected and/or maintained at a temperature that is at most about −78° C., at most about −76° C., at most about −74° C., at most about −72° C., at most about −70° C., at most about −68° C., at most about −66° C., at most about −64° C., at most about −62° C., at most about −60° C., at most about −58° C., at most about −56° C., at most about −54° C., at most about −52° C., at most about −50° C., at most about −48° C., at most about −46° C., at most about −44° C., at most about −42° C., at most about −40° C., at most about −38° C., at most about −36° C., at most about −34° C., at most about −32° C., at most about −30° C., at most about −28° C., at most about −26° C., at most about −24° C., at most about −22° C., at most about −20° C., at most about −18° C., at most about −16° C., at most about −14° C., at most about −12° C., at most about −10° C., at most about −8° C., at most about −6° C., at most about −4° C., at most about −2° C., at most about 0° C., at most about 2° C., at most about 4° C., at most about 6° C., at most about 8° C., at most about 10° C., at most about 12° C., at most about 14° C., at most about 16° C., at most about 18° C., at most about 20° C., at most about 22° C., at most about 24° C., at most about 26° C., at most about 28° C., at most about 30° C., at most about 32° C., or at most about 34° C. The biological sample can be collected and/or maintained at a temperature range between about −78° C. to about −20° C., about −78° C. to about 0° C., about −78° C. to about 4° C., about −78° C. to about 24° C., about −20° C. to about 0° C., about −20° C. to about 4° C., about-20° C. to about 24° C., about 0° C. to about 4° C., about 0° C. to about 24° C., or about 4° C. to about 24° C.

Biological samples (e.g., tissues samples, tissue biopsy samples, surgically removed tissue samples) used in methods described herein can be from a tumor of an individual. The tumor can be any type of cancerous tumor, including hematological malignancies, solid tumors, sarcomas, carcinomas, and other solid and non-solid tumors. Illustrative suitable cancers include, for example, adrenocortical carcinoma, anal cancer, appendiceal cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemias (e.g., acute lymphoblastic leukemia, acute myeloid leukemia, chronic lymphocytic leukemia, chronic myelogenous leukemia, hairy cell leukemia, myelodysplastic syndrome, prolymphocytic leukemia, large granular lymphocytic leukemia, adult T-cell leukemia, clonal eosinophilias), lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sézary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

Biological samples (e.g., tissue biopsy samples) used in methods described herein can be from primary tumors and/or secondary (e.g., metastatic) tumors of an individual. Any number of biological samples (e.g., of the two or more biological samples) can be from a primary tumor, such as at least 1, at least 2, at least 3, at least 4, at least 5, at least 6 at least 7, at least 8, at least 9, or at least 10. Any number of biological samples (e.g., of the two or more biological samples) can be from a secondary (e.g., a metastatic) tumor, such as at least 1, at least 2, at least 3, at least 4, at least 5, at least 6 at least 7, at least 8, at least 9, or at least 10. The tumor can be any stage of cancer including, but not limited to, stage 0, stage I, stage II, stage III, or stage IV. The tumor can be in remission (e.g., partial remission). The tumor can be a relapsed tumor (e.g., a tumor of a relapsed cancer). The tumor can have any grade including, but not limited to, X, 1, 2, 3, or 4. The tumor can be a recalcitrant tumor. The tumor can be resistant to therapy (e.g., resistant to chemotherapy, resistant to immunotherapy). The tumor can be susceptible to therapy (e.g., susceptible to chemotherapy, susceptible to immunotherapy, susceptible to radiotherapy).

Biological samples in methods described herein can be collected at any time point in relation to each other. Biological samples (e.g., two or more biological samples) can be collected at the same time (e.g., during the same surgical procedure, on the same day). Biological samples can be collected from an individual at different occasions that are temporally separated by any duration of time. The duration of time between collection of two biological samples can be about 1 minute, about 2 minutes, about 3 minutes, about 4 minutes, about 5 minutes, about 6 minutes, about 7 minutes, about 8 minutes, about 9 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 14 hours, about 16 hours, about 18 hours, about 20 hours, about 22 hours, about 1 day, about 2 days, about 3 days, about 4 days, about 5 days, about 6 days, about 7 days, about 8 days, about 9 days, about 10 days, about 12 days, about 14 days, about 16 days, about 18 days, about 20 days, about 22 days, about 24 days, about 26 days, about 28 days, about 30 days, about 1 month, about 2 months, about 3 months, about 4 months, about 5 months, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months about 22 months, about 23 months, or about 2 years. The duration of time between collection of two biological samples can be at least about 1 minute, at least about 5 minutes, at least about 10 minutes, at least about 15 minutes, at least about 30 minutes, at least about 45 minutes, at least about 1 hour, at least about 2 hours, at least about 4 hours, at least about 6 hours, at least about 8 hours, at least about 10 hours, at least about 12 hours, at least about 14 hours, at least about 16 hours, at least about 18 hours, at least about 20 hours, at least about 1 day, at least about 2 days, at least about 3 days, at least about 4 days, at least about 5 days, at least about 6 days, at least about 7 days, at least about 8 days, at least about 9 days, at least about 10 days, at least about 12 days, at least about 14 days, at least about 16 days, at least about 18 days, at least about 20 days, at least about 24 days, at least about 28 days, at least about 1 month, at least about 2 months, at least about 4 months, at least about 6 months, at least about 8 months, at least about 10 months, at least about 1 year, at least about 1.5 years, or at least about 2 years.

### Multi-Cancer Detection Test

Methods described herein can include the step of analyzing a biological sample from an individual at risk for incidence or recurrence of cancer using a multi-cancer detection (MCD) test to yield an MCD test result. Multi-cancer detection tests (e.g., multi-cancer early detection (MCED) tests) identify cancer (e.g., tumors) in an individual based on a biomarker (e.g., nucleic acid or protein profiles) of a biological sample from the individual. As discussed above, any type of biological sample can be used in methods described herein and analyzed by an MCD test.

Methods described herein can use any MCD test. The MCD test can analyze any property, biomarker, or component of a biological sample from an individual, including but not limited to nucleic acid methylation, nucleic acid mutation, nucleic acid fragmentation patterns, extracellular vesicle proteins, nucleic acid of circulating tumor cells, gene expression, circulating cancer antigens, and combinations of any of the foregoing. The MCD test can analyze gene expression by any method, such as RNA-seq, RT-PCR (reverse transcriptase polymerase chain reaction), or circulating tumor cell fluorescence in situ hybridization (ctc-FISH). The MCD test can quantify any of the above properties, biomarkers, or components of the biological sample. The MCD test can be a commercially available MCD test including but not limited to Adela™ (ADELA, INC.), Tr (ACE)™ (BIOLOGICAL DYNAMICS, INC.), BluestarMCED (BLUESTAR GENOMICS INC., CLEARNOTE HEALTH INC.), OverC™ (BURNING ROCK LTD), MIGPSai™ (CARIS LIFE SCIENCES, INC.), DELFI (DELFI DIAGNOSTICS, INC.), cf Methyl-Seq (EARLYDIAGNOSTICS INC.), CancerSEEK (EXACT SCIENCES CO.), FMBT (FREENOME HOLDINGS, INC.), Galleri® multi-cancer test (GRAIL, LLC; ILLUMINA, INC.), LungLB (LUNGLIFEAI), Signatera™ (NATERA), Sentinel-10TM (PRECISION EPIGENOMICS), OneTEST (20/20 GENE SYSTEMS), PanSEER (SINGLERA GENOMICS), DEEPGEN (QUANTGENE INC.), and combinations of any of the foregoing.

Any type of nucleic acid can be analyzed by an MCD test including, but not limited to cell free DNA (cfDNA), circulating tumor DNA (ctDNA), cell free RNA (cfRNA) (e.g., cell free microRNA (cf-miRNA), cell free messenger RNA (cf-mRNA)), circulating tumor RNA (ctRNA), and combinations of any of the foregoing. The nucleic acid analyzed by the MCD test can contain any type of nucleotide, including but not limited to a canonical nucleotide of the genetic code (e.g., adenosine, cytidine, guanosine, thymidine, uridine, 2′-deoxyadenosine, 2′-deoxycytidine, 2′-deoxyguanosine), an epigenetically modified nucleotide (e.g., a methylated nucleotide (e.g., N°-methyladenosine, 5-methylcytidine, 7-methylguanosine), a hydroxymethylated nucleotide (e.g., 5-hydroxymethylcytidine), a formylated nucleotide (e.g., 5-formylcytidine), a carboxylated nucleotide (e.g., 5-carboxycytidine), an oxidized nucleotide (e.g., 7,8-dehydro-8-oxoguanosine), a glycosidic C-C linked nucleotide (e.g., pseudouridine), an acetylated nucleotide (e.g., N⁴-acetylcytidine), and the like), a chemical nucleotide adduct (e.g., 7-(1-hydroxy-3-buten-2-yl) guanosine, 8-(4-aminobiphenyl) guanosine), and combinations of any of the foregoing. In some embodiments, the MCD test comprises nucleic acid sequencing of a biological sample from an individual at risk for incidence or recurrence of cancer. The MCD test can analyze a nucleic acid using a panel of genes or sequences. The MCD test can include a panel of known genes or mutations (e.g., known genes or mutations associated with cancer progression, cancer development, or cancer incidence; oncogenes; cancer suppressor genes). The MCD test can analyze a nucleic acid using unbiased sequencing techniques (e.g., can sequence any nucleic acid present in a biological sample).

### Determination of Neoantigens and Generation of Neoantigen Immunogenic Compositions

Methods described herein can determine, score, and/or select neoantigens for generating a neoantigen immunogenic composition (e.g., a cancer vaccine). Any method of determining, scoring, and selecting neoantigens can be used. Examples of suitable methods include those described by WO2022/159176A1, US20230197192A1, US20230173045A1, and U.S. Pat. No. 20,220,093209A1, the entire contents of each of which are incorporated herein.

Neoantigens are self-antigens generated by tumor cells due to genomic mutations or dysregulated RNA splicing. Neoantigens identified by sequencing in methods described herein can be in the form of any sequencing results including, but not limited to, sequence reads (DNA sequence reads, RNA sequence reads), sequence variants (e.g., peptide-modifying sequence variants), encoded peptides, or combinations of the foregoing. The neoantigens can be any type of peptide, including but not limited to long peptides, short peptides, or combinations thereof. In some embodiments, the one or more neoantigens are peptides that are long peptides, short peptides, or a combination thereof. Neoantigens of methods described herein can be tumor-specific (e.g., neoantigens only present in a cancer or tumor and not in healthy or germ-line cells of a subject).

As used herein, the terms “short identified neoantigen peptide” and “short peptide” can refer to a peptide with an amino acid length that is between about 3 to about 15, about 3 to about 14, about 3 to about 13, about 3 to about 12, about 3 to about 11, about 3 to about 10, about 3 to about 9, about 3 to about 8, about 3 to about 7, about 3 to about 6, about 3 to about 5, about 3 to about 4, about 4 to about 15, about 5 to about 15, about 6 to about 15, about 7 to about 15, about 8 to about 15, about 9 to about 15, about 10 to about 15, about 11 to about 15, about 12 to about 15, about 13 to about 15, about 14 to about 15, about 8 to about 11, about 8 to about 10, about 8 to about 9, about 9 to about 11, about 10 to about 11, about 7 to about 11, about 6 to about 11, about 5 to about 11, about 4 to about 11, about 3 to about 11, about 8 to about 12, about 8 to about 13, or about 8 to about 14. “Short identified neoantigen peptide” and “short peptide” can refer to a peptide with an amino acid length that is about 3, about 4, about 5, about 6, about 7, about 8, about 9, about 10, about 11, about 12, about 13, about 14, or about 15. Short identified neoantigen peptide” and “short peptide” can refer to a peptide with an amino acid length that is at most about 15, at most about 14, at most about 13, at most about 12, at most about 11, at most about 10, at most about 9, at most about 8, at most about 7, at most about 6, at most about 5, at most about 4, at most about 3, or at most about 2. As used herein, the terms “long identified neoantigen peptide” and “long peptide” can refer to a peptide with an amino acid length that is between about 13 and about 30, about 13 and about 29, about 13 and about 28, about 13 and about 27, about 13 and about 26, about 13 and about 25, about 13 and about 24, about 13 and about 23, about 13 and about 22, about 13 and about 21, about 13 and about 20, about 13 and about 19, about 13 and about 18, about 13 and about 17, about 13 and about 16, about 13 and about 15, about 13 and about 14, about 14 and about 30, about 15 and about 30, about 16 and about 30, about 17 and about 30, about 18 and about 30, about 19 and about 30, about 20 and about 21, about 22 and about 30, about 23 and about 30, about 24 and about 30, about 25 and about 30, about 26 and about 30, about 27 and about 30, about 28 and about 30, about 29 and about 30, or about 13 and about 25. “Long identified neoantigen peptide” and “long peptide” can refer to a peptide with an amino acid length that is about 13, about 14, about 15, about 16, about 17, about 18, about 19, about 20, about 21, about 22, about 23, about 24, about 25, about 26, about 27, about 28, about 29, or about 30. “Long identified neoantigen peptide” and “long peptide” can refer to a peptide with an amino acid length that is at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, or at least about 30.

Methods described herein can include steps to determine neoantigens appropriate for inclusion in a neoantigen immunogenic composition. Methods can further include the step of determining the human leukocyte antigen (HLA) or major histocompatibility complex (MHC) type for the individual at risk for incidence or recurrence of cancer. The HLA type can be determined through any method including but not limited to nucleic acid sequencing (e.g., RNA sequencing, DNA sequencing, complementary (cDNA) sequencing), immunoassay (e.g., enzyme linked immunosorbent assay (ELISA)), mass spectrometry, flow cytometry, real-time PCR, or combinations of any of the foregoing. Methods can include the step of scoring neoantigens of a biological sample from the individual at risk for incidence or recurrence of cancer. The biological sample used for identifying or scoring neoantigens can be the same sample used for the MCD test. The biological sample used for identifying or scoring neoantigens can be a different (e.g., subsequent, new) biological sample from an individual at risk for incidence or recurrence of cancer that is not the same as the biological sample used in the MCD test. Methods can include the step of selecting neoantigens of a biological sample for inclusion in the neoantigen immunogenic composition. Any number of neoantigens can be selected for inclusion in the neoantigen immunogenic composition. The number of neoantigens selected can be at most 20, at most 19, at most 18, at most 17, at most 16, at most 15, at most 14, at most 13, at most 12, at most 11, at most 10, at most 9, at most 8, at most 7, at most 6, at most 5, at most 4, at most 3, at most 2, or at most 1. The number of neoantigens selected can be at least 20, at least 19, at least 18, at least 17, at least 16, at least 15, at least 14, at least 13, at least 12, at least 11, at least 10, at least 9, at least 8, at least 7, at least 6, at least 5, at least 4, at least 3, at least 2, or at least 1. In some embodiments, the method further comprises the steps of determining the HLA type for an individual at risk for incidence or recurrence of cancer, scoring neoantigens of a biological sample from an individual at risk for incidence or recurrence of cancer, selecting neoantigens of a biological sample for inclusion in a neoantigen immunogenic composition, and/or generating the neoantigen immunogenic composition.

Neoantigens suitable for use in generating a neoantigen immunogenic composition can be determined by sequencing nucleic acids from a biological sample of an individual. In certain embodiments, the MCD test comprises nucleic acid sequencing of a biological from an individual. In such embodiments, the MCD test result (e.g., the sequencing result of the MCD test) can provide raw data for determining, scoring, and/or selecting neoantigens. In some embodiments, the same biological sample used for the MCD test is subjected to sequencing (e.g., deeper sequencing than the MCD test, a subsequent or additional sequencing step) and analysis to identify neoantigens. Sequencing can be any type of sequencing including but not limited to whole genome sequencing, shotgun metagenomic sequencing, whole exome sequencing, next-generation sequencing (NGS), cancer personalized profiling by deep sequencing (CAPP-Seq), tagged-amplicon deep sequencing (Tam-Seq), or combinations of any of the foregoing. In certain embodiments, a new biological sample (e.g., a different biological sample than that analyzed by the MCD test) is obtained from the individual and subjected to sequencing and analysis to identify neoantigens. In some embodiments, the method includes the step of sequencing and analyzing of the biological sample from the individual at risk for incidence or recurrence of cancer or a new biological sample from the individual at risk for incidence or recurrence of cancer to identify neoantigens to be included in the neoantigen immunogenic composition.

Neoantigen peptides can be predicted by any method. One suitable method for predicting neoantigen peptides is analysis (via an algorithm) of protein variants expressed and isoforms translated from population-level statistics. Another suitable method for predicting neoantigen peptides is by translation of RNA sequencing data (e.g., RNA sequencing data that overlaps each somatic variant). Another suitable method for predicting neoantigen peptides is by transcription of DNA sequences (e.g., cfDNA sequences) into RNA and translation of the resulting RNA sequencing data.

Any strategy or method can be used to reduce the background noise of nucleic acids or proteins originating from healthy non-cancerous cells, and to increase the signal to noise ratio of nucleic acids or proteins from cancerous cells (e.g., sequencing variants that encode for neoantigens). One suitable method for increasing the signal to noise ratio of sequencing variants is by sequencing a biological sample to yield non-cancer sequencing results (e.g., sequencing results from non-cancerous cells, sequencing results lacking oncogenic somatic mutations) and cancer sequencing results (e.g., sequencing results from cancer cells, sequencing results containing somatic mutations). In this method, the non-cancer sequencing results can be subtracted (e.g., eliminated, deleted) from the cancer sequencing results to improve the signal to noise ratio for variant calling of neoantigens. The non-cancer sequencing results and the cancer sequencing results can be obtained from the same biological sample. For example, the cancer sequencing results and the non-cancer sequencing results can both be obtained from the same liquid biopsy sample (e.g., the same blood sample). Continuing this example, the liquid biopsy sample can be a blood sample and the non-cancer sequencing results are obtained from sequencing the genetic material of white blood cells, while the cancer sequencing results are obtained from sequencing cell free DNA (cfDNA). The non-cancer sequencing results and the cancer sequencing results can be obtained from two different biological samples. For example, the non-cancer sequencing results can be obtained from a tissue biopsy (e.g., a biopsy of healthy, non-cancerous tissue) and the cancer sequencing results can be obtained from tissue resection of a tumor. Another method to increase the signal to noise is to use unique molecular identifiers (UMI) and oversample during sequencing to facilitate error correction. In some embodiments, the method comprises the step of sequencing nucleic acids of a biological sample to yield non-cancer sequencing results and cancer sequencing results, wherein the non-cancer sequencing results are subtracted from the cancer sequencing results to improve the signal to noise ratio for variant calling of neoantigens.

Any number of biological samples can be sequenced and analyzed to identify, determine, score, and/or select neoantigens. The number of biological samples can be about 1, about 2, about 3, about 4, about 5, about 6, about 7, about 8, about 9, about 10, about 11, about 12, about 13, about 14, about 15, about 16, about 17, about 18, about 19, or about 20. The number of biological samples can be at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20. The number of biological samples can be between about 1 to about 20, about 1 to about 19, about 1 to about 18, about 1 to about 17, about 1 to about 16, about 1 to about 15, about 1 to about 14, about 1 to about 13, about 1 to about 12, about 1 to about 11, about 1 to about 10, about 1 to about 9, about 1 to about 8, about 1 to about 7, about 1 to about 6, about 1 to about 5, about 1 to about 4, about 1 to about 3, about 1 to about 2, about 2 to about 20, about 3 to about 20, about 4 to about 20, about 5 to about 20, about 6 to about 20, about 7 to about 20, about 8 to about 20, about 9 to about 20, about 10 to about 20, about 11 to about 20, about 12 to about 20, about 13 to about 20, about 14 to about 20, about 15 to about 20, about 16 to about 20, about 17 to about 20, about 18 to about 20, about 19 to about 20, about 2 to about 19, about 2 to about 18, about 2 to about 17, about 2 to about 16, about 2 to about 15, about 2 to about 14, about 2 to about 13, about 2 to about 12, about 2 to about 11, about 2 to about 10, about 2 to about 9, about 2 to about 8, about 2 to about 7, about 2 to about 6, about 2 to about 5, about 2 to about 4, about 2 to about 3, about 3 to about 19, about 3 to about 18, about 3 to about 17, about 3 to about 16, about 3 to about 15, about 3 to about 14, about 3 to about 13, about 3 to about 12, about 3 to about 11, about 3 to about 10, about 3 to about 9, about 3 to about 8, about 3 to about 7, about 3 to about 6, about 3 to about 5, about 3 to about 4, about 4 to about 19, about 4 to about 18, about 4 to about 17, about 4 to about 16, about 4 to about 15, about 4 to about 14, about 4 to about 13, about 4 to about 12, about 4 to about 11, about 4 to about 10, about 4 to about 9, about 4 to about 8, about 4 to about 7, about 4 to about 6, or about 4 to about 5.

Sequencing results obtained by methods described herein (e.g., an MCD test result, a sequencing result, a subsequent sequencing result) can be used to determine peptide sequences that are encoded by nucleic acids (e.g., encoded by DNA, encoded by RNA). The nucleic acid sequenced to obtain a sequencing result can be any type of nucleic acid, including but not limited to RNA, DNA, cfDNA, cfRNA (cell-free RNA), circulating tumor DNA (ctDNA), circulating tumor RNA (ctRNA), or combinations of any of the foregoing. The sequencing result can be in any format including but not limited to CRAM format, General Feature Format (e.g., GFF3), FASTA format, FASTQ format, NeXML format, Nexus format, Pileup format, Sequence Alignment Map (SAM) format, Stockholm format, Variant Call Format (VCF) format, genomic VCF (gVCF) format, or a combination of any of the foregoing. In some embodiments, the sequencing result is circulating tumor DNA and the format of the sequencing result is VCF format, wherein the VCF file contains tumor-specific somatic variants found in the biological sample. In some embodiments, the sequencing result is obtained from sequencing healthy and tumor tissue and the format is separate FASTQ files of the sequencing results from healthy and tumor tissue. The resulting peptide sequences can be analyzed to determine whether the encoded peptide is a neoantigen based on the presence of sequence variants (e.g., sequence variants in the peptide amino acid sequence compared to the healthy cells or healthy tissue of a subject, sequence variants in the peptide amino acid sequence compared to a reference genome). Neoantigens (e.g., neoantigen peptides) identified by methods described herein can be analyzed to determine a predicted immunogenicity based on any factor including, but not limited to whether the neoantigen is immunogenic (e.g., whether a neoantigen can elicit an immune response in a subject, prediction of major histocompatibility complex (MHC) binding affinity, whether peptide is predicted to be presented on a cell surface by an MHC molecule), whether the tumor expresses an amount of neoantigen sufficient to elicit an immune response, whether the neoantigen is expressed on a sufficient fraction of the tumor cells, relative or absolute amount of nucleic acids encoding for the neoantigen present in the sample (e.g., present in the total cfDNA) or a combination thereof. Any methodology for predicting immunogenicity of a neoantigen can be used in methods described herein. For example, the number of sequenced variants (e.g., DNA sequence variants, RNA sequence variants, encoded peptide variants) can be used to predict the expression of a neoantigen in a biological sample and infer the expression in the originating tissue of the subject (e.g., in a tumor of the subject). For several examples of predicting immunogenicity of a neoantigen, see WO2022/159176A1, US20230197192A1, US20230173045A1, and US20220093209A1, all of which are hereby incorporated by reference in their entireties. The predicted immunogenicity can be used to score a neoantigen. Neoantigens can be scored by any parameter of the neoantigen. For example, the neoantigens can be scored by the counts of nucleic acids (e.g., DNA, RNA) encoding the neoantigen (e.g., RNA transcripts per million (RNA TPM), RNA expression values). For example, neoantigens can be scored by cellular prevalence of the neoantigen (e.g., cells expressing the neoantigen as determined by single cell sequencing of circulating tumor cells or ctc-FISH staining, cells expressing the neoantigen protein as determined by cell cytometry of circulating tumor cells). Neoantigen peptides can be scored by binding affinity (e.g., experimental binding affinity, predicted binding affinity) to a major histocompatibility complex (MHC), frequency of mutant alleles, gene expression, C-terminal cleavage affinity, transporter associated with antigen processing (TAP) transport of the neoantigen peptide, or combinations of any of the foregoing. The scores of predicted immunogenicity can be ranked (e.g., neoantigens can be ranked in order of predicted immunogenicity). The scored neoantigens can be further selected for inclusion (e.g., inclusion of a neoantigen peptide, inclusion of a nucleic acid encoding for a neoantigen peptide) in a neoantigen immunogenic composition (e.g., a cancer vaccine). The scored neoantigens can be further selected for exclusion (e.g., exclusion of a neoantigen peptide, exclusion of a nucleic acid encoding for a neoantigen peptide) in a neoantigen immunogenic composition (e.g., a cancer vaccine).

Sequence variants (e.g., mutations) identified from biological samples (e.g., liquid biopsy samples, blood samples) can be from non-cancer cells or cancer cells (e.g., ctDNA, ctRNA). Any method to distinguish non-cancer cell and cancer cell sequence variants can be used in methods described herein. For example, multiple sequence variants can be screened simultaneously to improve the probability of detecting circulating tumor nucleic acids (e.g., ctDNA, ctRNA). As another example, the probability of detecting circulating tumor nucleic acids can be increased based on detection of epigenetic modifications (e.g., methylation profile, including 5-methylcytosine (5mC) and/or 5-hydroxymethylcytosine (5hmC)) of the nucleic acids (e.g., cfDNA). As another example, the probability of detecting circulating tumor nucleic acids can be increased based on the fragmentation pattern (e.g., fragment lengths, fragment start positions, fragment end nucleotide motifs) of nucleic acids (e.g., cfDNA). As another example, the probability of detecting circulating tumor nucleic acids can be increased based on the mutational signature profiles of known human carcinogens, existence of co-mutations, and/or evolutionary cancer signatures.

Methods described herein can include the step of generating a neoantigen immunogenic composition. The neoantigen immunogenic composition (e.g., cancer vaccine) can be any immunogenic composition, such as those described in WO2022/170067 A1, US2023/0173045 A1, WO2022/159176 A1, WO2022/251034 A1, or US2023/0173046 A1, the entirety of each of which are incorporated by reference herein. The neoantigen immunogenic composition can comprise or encode neoantigens (e.g., one or more neoantigens) scored for predicted immunogenicity by any method including by the methods described herein. The neoantigen immunogenic composition can contain neoantigens in any form, including but not limited to peptides (e.g., long identified peptides, short identified peptides, synthesized peptides, isolated peptides), RNA sequences encoding for peptides (e.g., mRNA, mRNA containing unnatural nucleotides (e.g., pseudouridine, N¹-methylpseudouridine, 7-methylguanosine, N⁶-methyladenosine, 2′-O-methyl nucleotide), mRNA containing inverted nucleotides, or combinations thereof), DNA sequences encoding for peptides (e.g., plasmid DNA, viral DNA), or combinations thereof. Neoantigen immunogenic compositions containing nucleic acids (e.g., RNA, DNA) encoding for peptides can be in the form of a virus (e.g., an adenovirus, lentivirus, fowl pox, vaccinia, self-replicating alphavirus, Maraba virus).

A nucleic acid (e.g., RNA, DNA) of a neoantigen immunogenic composition can encode for any number of peptide antigens. The number of peptide antigens encoded by a nucleic acid can be about 1, about 2, about 3, about 4, about 5, about 6, about 7, about 8, about 9, about 10, about 11, about 12, about 13, about 14, about 15, about 16, about 17, about 18, about 19, or about 20. The number of peptide antigens encoded by a nucleic acid can be at most about 1, at most about 2, at most about 3, at most about 4, at most about 5, at most about 6, at most about 7, at most about 8, at most about 9, at most about 10, at most about 11, at most about 12, at most about 13, at most about 14, at most about 15, at most about 16, at most about 17, at most about 18, at most about 19, or at most about 20. In some embodiments, the method includes the step of generating a neoantigen immunogenic composition, wherein the neoantigen immunogenic composition comprises or encodes for one or more neoantigens scored for the predicted immunogenicity by the method.

A neoantigen immunogenic composition can contain or encode for any number of neoantigens (e.g., neoantigen proteins, DNA sequences encoding for neoantigen proteins, RNA sequences encoding for neoantigen proteins). A neoantigen immunogenic composition can contain or encode for a number of neoantigens that is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30. The number of neoantigens contained or encoded by a neoantigen immunogenic composition can be one or more, two or more, three or more, four or more, five or more, six or more, seven or more, eight or more, nine or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, or 29 or more, or 30 or more. The number of neoantigens contained or encoded by a neoantigen immunogenic composition can be at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, or at least about 30. The number of neoantigens contained or encoded by a neoantigen immunogenic composition can be between about 1 to about 50, about 2 to about 50, about 3 to about 50, about 4 to about 50, about 5 to about 50, about 6 to about 50, about 7 to about 50, about 8 to about 50, about 9 to about 50, about 10 to about 50, about 11 to about 50, about 12 to about 50, about 13 to about 50, about 14 to about 50, about 15 to about 50, about 16 to about 50, about 17 to about 50, about 18 to about 50, about 19 to about 50, about 20 to about 50, about 22 to about 50, about 24 to about 50, about 26 to about 50, about 28 to about 50, about 30 to about 50, about 33 to about 50, about 36 to about 50, about 40 to about 50, about 45 to about 50, about 1 to about 45, about 1 to about 40, about 1 to about 36, about 1 to about 33, about 1 to about 30, about 1 to about 28, about 1 to about 26, about 1 to about 24, about 1 to about 22, about 1 to about 20, about 1 to about 18, about 1 to about 16, about 1 to about 14, about 1 to about 12, about 1 to about 10, about 1 to about 9, about 1 to about 8, about 1 to about 7, about 1 to about 6, about 1 to about 5, about 1 to about 4, about 1 to about 3, about 1 to about 2, about 2 to about 14, about 2 to about 12, about 2 to about 10, about 2 to about 8, about 2 to about 6, about 2 to about 4, about 3 to about 14, about 3 to about 12, about 3 to about 10, about 3 to about 8, about 3 to about 6, about 3 to about 4, about 4 to about 14, about 4 to about 12, about 4 to about 10, about 4 to about 8, about 4 to about 6, about 5 to about 14, about 5 to about 12, about 5 to about 10, about 5 to about 8, about 5 to about 6, about 6 to about 14, about 6 to about 12, about 6 to about 10, about 6 to about 8, about 7 to about 14, about 7 to about 12, about 7 to about 10, about 7 to about 8, about 8 to about 14, about 8 to about 12, about 8 to about 10, about 9 to about 14, about 9 to about 12, about 9 to about 10, about 10 to about 14, about 10 to about 12, about 11 to about 14, about 11 to about 12, about 12 to about 14, or about 13 to about 14.

Neoantigen immunogenic compositions described herein may comprise up to about 50 neoantigen long peptides and/or short peptides. The neoantigen immunogenic composition may comprise about 10 to about 20 neoantigen long peptides and/or short peptides. In some embodiments, the neoantigen immunogenic composition comprises about 19 neoantigen long peptides and/or short peptides.

The neoantigen immunogenic composition may comprise at least about 2 or more neoantigen long peptides. The neoantigen immunogenic composition may comprise about 2 to about 18 neoantigen long peptides. The neoantigen immunogenic composition can comprise at least about 10 to about 15 neoantigen long peptides. The neoantigen immunogenic composition may comprise at least about 2 or more neoantigen short peptides. The neoantigen immunogenic composition may comprise at least about 2 to about 10 neoantigen short peptides.

The selected neoantigens can be divided into pools for separate administration of neoantigen immunogenic compositions to the individual at risk for incidence or recurrence of cancer in need thereof. The pools of neoantigens can contain any number of neoantigens. The number of neoantigens in a pool can be about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50. The number of neoantigens in a pool can be at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50. The number of neoantigens in a pool can be at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, at most 40, at most 41, at most 42, at most 43, at most 44, at most 45, at most 46, at most 47, at most 48, at most 49, or at most 50. The number of neoantigens in a pool can be about 1 to about 30, 1 to about 28, 1 to about 26, 1 to about 24, 1 to about 22, 1 to about 20, about 1 to about 18, about 1 to about 16, about 1 to about 14, about 1 to about 12, about 1 to about 10, about 1 to about 9, about 1 to about 8, about 1 to about 7, about 1 to about 6, about 1 to about 5, about 1 to about 4, about 1 to about 3, or about 1 to about 2. As an example, three peptide pools of the neoantigen immunogenic composition may comprise about 5 neoantigen long peptides and/or short peptides and one peptide pool may comprise 4 neoantigen long peptides and/or short peptides and a helper peptide. Each peptide pool may comprise different neoantigen long peptides and/or short peptides. Neoantigen long peptides may be about 15 to about 30 amino acids in length. Neoantigen short peptides may be about 5 to about 15 amino acids in length.

The selected neoantigens for the immunogenic composition can be split into any number of pools. The number of pools can be about 1, about 2, about 3, about 4, about 5, about 6, about 7, about 8, about 9, about 10, about 11, about 12, about 13, about 14, about 15, about 16, about 17, about 18, about 19, or about 20. The number of pools can be at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20. The number of pools can be at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, or at most 20. The number of pools can be between about 1 to about 2, about 1 to about 3, about 1 to about 4, about 1 to about 5, about 1 to about 6, about 1 to about 7, about 1 to about 8, about 1 to about 9, about 1 to about 10, about 2 to about 10, about 3 to about 10, about 4 to about 10, about 5 to about 10, about 6 to about 10, about 7 to about 10, about 8 to about 10, about 9 to about 10, about 2 to about 3, about 2 to about 4, about 2 to about 5, about 2 to about 6, about 2 to about 7 about 2 to about 8, about 2 to about 9, about 2 to about 10, about 3 to about 4, about 3 to about 5, about 3 to about 6, about 3 to about 7, about 3 to about 8, about 3 to about 9, about 3 to about 10, about 4 to about 5, about 4 to about 6, about 4 to about 7, about 4 to about 8, about 4 to about 9, or about 4 to about 10.

The neoantigen immunogenic composition can comprise at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, at least about 50 or more neoantigen peptides (e.g., neoantigen long peptides and/or short peptides). The neoantigen immunogenic composition can comprise up to about 100 neoantigen peptides. The neoantigen immunogenic composition can contain about 1-5 neoantigens, 1-10 neoantigens, about 1-15 neoantigens, about 4-10 neoantigens, about 4-15 neoantigens, about 10-20 neoantigens, about 10-30 neoantigens, about 10-40 neoantigens, about 10-50 neoantigens, about 10-60 neoantigens, about 10-70 neoantigens, about 10-80 neoantigens, about 10-90 neoantigens, or about 10-100 neoantigens. For example, the neoantigen immunogenic composition can comprise about 10, about 11, about 12, about 13, about 14, about 15, about 16, about 17, about 18, about 19, or about 20 neoantigens. In some embodiments, the neoantigen immunogenic composition can comprise about 19 neoantigens. Each of the neoantigens in the neoantigen immunogenic composition can be different.

The neoantigen immunogenic composition described herein can further comprise an adjuvant. Adjuvants are any substance whose admixture into a neoantigen immunogenic composition increases, or otherwise enhances and/or boosts, the immune response to a tumor-specific neoantigen, but when the substance is administered alone does not generate an immune response to a tumor-specific neoantigen. The adjuvant can generate an immune response to the neoantigen and does not produce an allergy or other adverse reaction. It is contemplated herein that adjuvant can be administered before, together, concomitantly with, or after administration of the neoantigen immunogenic composition.

Adjuvants can enhance an immune response by several mechanisms including, e.g., lymphocyte recruitment, stimulation of B and/or T cells, and stimulation of macrophages. When a neoantigen immunogenic composition described herein comprises adjuvants or is administered together with one or more adjuvants, the adjuvants that can be used include, but are not limited to, mineral salt adjuvants or mineral salt gel adjuvants, particulate adjuvants, microparticulate adjuvants, mucosal adjuvants, immunostimulatory adjuvants, or combinations thereof. Examples of adjuvants include, but are not limited to, aluminum salts (alum) (such as aluminum hydroxide, aluminum phosphate, and aluminum sulfate), 3 De-O-acylated monophosphoryl lipid A (MPL) (see, GB 2220211), MF59 (Novartis), AS03 (Glaxo SmithKline), AS04 (Glaxo SmithKline), polysorbate 80 (Tween 80; ICL Americas, Inc.), imidazopyridine compounds (see, International Application No. PCT/US2007/064857, published as International Publication No. WO2007/109812, published as U.S. Pat. App. publication No. US20090232844A1 and corresponding to U.S. Pat. No. 8,063,063), imidazoquinoxaline compounds (see, International Application No. PCT/US2007/064858, published as International Publication No. WO2007/109813, published as U.S. Pat. App. publication No. US20090311288A1 and corresponding to U.S. Pat. No. 8,173,657) and saponins, such as QS21 (see, Kensil et al, in Vaccine Design: The Subunit and Adjuvant Approach (eds. Powell & Newman, Plenum Press, NY, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see, Stoute et al, N. Engl. J. Med. 336, 86-91 (1997)). CpG immunostimulatory oligonucleotides have also been reported to enhance the effects of adjuvants in a vaccine setting. Other TLR binding molecules such as RNA binding TLR 7, TLR 8 and/or TLR 9 may also be used. Other examples of useful adjuvants include, but are not limited to, chemically modified CpGs (e.g., CpR, Idera), Poly (I:C) (e.g., polyi:CI2U), poly ICLC, non-CpG bacterial DNA or RNA as well as immunoactive small molecules and antibodies such as cyclophosphamide, sunitmib, bevacizumab, Celebrex (celecoxib), NCX-4016, sildenafil, tadalafil, vardenafil, sorafenib, XL-999, CP-547632, pazopanib, ZD2171, AZD2171, ipilimumab, tremelimumab, and SC58175, which may act therapeutically and/or as an adjuvant.

Neoantigen immunogenic composition described herein can be administered to a subject that has not been diagnosed with cancer, has been diagnosed with cancer, is already suffering from cancer, has recurrent cancer (e.g., relapse), or is at risk of developing cancer. A neoantigen immunogenic composition can be administered to a subject that is resistant to other forms of cancer treatment (e.g., chemotherapy, immunotherapy, or radiation). Neoantigen immunogenic composition can be administered to the subject prior to other standard of care cancer therapies (e.g., chemotherapy, immunotherapy, or radiation). Neoantigen immunogenic composition can be administered to the subject concurrently, after, or in combination to other standard of care cancer therapies (e.g., chemotherapy, immunotherapy, or radiation). In some embodiments, the method includes the step of based on the MCD test result (e.g., a positive MCD test result) and optionally the risk stratification parameter, administering a neoantigen immunogenic composition to the individual at risk for incidence or recurrence of cancer in need thereof.

Neoantigen immunogenic composition described herein can be administered to a subject in an amount sufficient to elicit an immune response to the tumor-specific neoantigen and to destroy, or at least partially arrest, symptoms and/or complications. In embodiments, the neoantigen immunogenic composition can provide a long-lasting immune response. A long-lasting immune response can be established by administering a boosting dose of the neoantigen immunogenic composition to the subject. The immune response to the neoantigen immunogenic composition can be extended by administering to the subject a boosting dose. In embodiments, at least one, at least two, at least three or more boosting doses can be administered to abate the cancer. A first boosting dose may increase the immune response by at least 50%, at least 100%, at least 200%, at least 300%, at least 400%, at least 500%, or at least 1000%. A second boosting dose may increase the immune response by at least 50%, at least 100%, at least 200%, at least 300%, at least 400%, at least 500%, or at least 1000%. A third boosting dose may increase the immune response by at least 50%, at least 100%, at least 200%, at least 300%, at least 400%, at least 500%, or at least 1000%.

An amount adequate to elicit an immune response is defined as a “therapeutically effective dose.” Amounts effective for this use will depend on, e.g., the composition, the manner of administration, the stage and severity of the disease being treated, the weight and general state of health of the individual, and the judgment of the prescribing physician. It should be kept in mind that neoantigen immunogenic composition can generally be employed in serious disease states, that is, life-threatening or potentially life-threatening situations, especially when the cancer has metastasized. In such cases, in view of the minimization of extraneous substances and the relative nontoxic nature of a neoantigen, it is possible and can be felt desirable by the treating physician to administer substantial excesses of neoantigen immunogenic compositions (e.g., cancer vaccines).

### Individuals at Risk for Incidence or Recurrence of Cancer

Methods described herein can be used on any subject in need thereof. The term “individual” as used herein refers to any mammalian subject in need of treatment by methods described herein. The mammalian subject can be any mammal including but not limited to a dog, a sheep, a goat, a cow, a horse, a cat, a rodent (e.g., a mouse, a rat, a hamster), a monkey, a non-human primate, a human, and the like. In certain embodiments, the subject in need thereof is an individual at risk for incidence or recurrence of cancer that is a human.

The individual at risk for incidence or recurrence of cancer can have a past history of cancer. The individual at risk for incidence or recurrence of cancer can be a cancer survivor (e.g., the individual had cancer that is currently in remission, the individual has no current cancer diagnosis). Cancer survivors can have a high incidence rate of future cancer (e.g., cancer recurrence). For example, breast cancer survivors that are 10-years disease-free have been found to have a cumulative breast cancer recurrence incidence of 16.6%. (Pedersen, R. N., et al. The Incidence of Breast Cancer Recurrence 10-32 Years After Primary Diagnosis. J. Natl. Cancer Inst. (2022) 114, 3, 391-399.) The cancer survivor can have minimal residual disease (MRD). The cancer survivor can be currently monitored for relapse or recurrence of cancer.

Individuals in need of treatment by methods described herein can have minimal residual disease (MRD, also known as measurable residual disease). For example, an individual suitable for practicing methods described herein can have a positive MRD test result. Individuals with MRD have a very small number of cancer cells remaining in their body during or following an antineoplastic treatment (e.g., chemotherapeutic treatment, immunotherapeutic treatment). Detection of MRD in an individual can indicate the likelihood of recurrence of cancer (e.g., recurrence of a cancer in an individual that is a cancer survivor). Detection of MRD can also indicate the efficacy of a current or prior antineoplastic treatment. For example, detection of MRD in an individual may indicate that additional doses of a therapeutic or increased dosage amount is necessary. An individual with MRD can be subject to a conventional standard of treatment that is no treatment (e.g., “watch and wait,” “watchful waiting”, “active surveillance”). An individual in need of treatment by methods described herein can have an MRD that is detected by any method, including but not limited to polymerase chain reaction (PCR, e.g., real-time PCR, quantitative real-time PCR, digital PCR), flow cytometry (e.g., multi-parametric flow cytometry (MFC)), nucleic acid sequencing (e.g., next generation sequencing (NGS), cancer personalized profiling by deep sequencing (CAPP-Seq), tagged-amplicon deep sequencing (Tam-Seq)), imaging (e.g., microscopy, immunohistochemistry), epigenetic marker detection (e.g., detection of nucleic acid methylation), or a combination of any of the foregoing. As discussed with respect to MCD tests, MRD tests can be conducted on any biological sample from an individual, such as any type of biological sample described herein. In some embodiments, the MCD test is a test for MRD and/or detects MRD in an individual.

Cancer survivors can be at any stage of remission or treatment. The cancer survivor can be disease free (e.g., cancer free) for any length of time prior to practicing methods described herein, including but not limited to 1 day, 2 days, 3 days, 4 days, 5 days, 6 days, 1 week, 2 weeks, 3 weeks, 4 weeks, 1 month, 2 months, 3 months, 4 months, 5 months, 6 months, 7 months, 8 months, 9 months, 10 months, 11 months, 1 year, 1.5 years, 2 years, 3 years, 4 years, 5 years, 6 years, 7 years, 8 years, 9 years, 10 years, 11 years, or 12 years. The cancer survivor can be disease free (e.g., cancer free) for at least 1 day, at least 2 days, at least 3 days, at least 4 days, at least 5 days, at least 6 days, at least 1 week, at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, at least 1 year, at least 1.5 years, at least 2 years, at least 3 years, at least 4 years, at least 5 years, at least 6 years, at least 7 years, at least 8 years, at least 9 years, at least 10 years, at least 11 years, or at least 12 years. The cancer survivor can be disease free (e.g., cancer free) for at most 1 day, at most 2 days, at most 3 days, at most 4 days, at most 5 days, at most 6 days, at most 1 week, at most 2 weeks, at most 3 weeks, at most 4 weeks, at most 1 month, at most 2 months, at most 3 months, at most 4 months, at most 5 months, at most 6 months, at most 7 months, at most 8 months, at most 9 months, at most 10 months, at most 11 months, at most 1 year, at most 1.5 years, at most 2 years, at most 3 years, at most 4 years, at most 5 years, at most 6 years, at most 7 years, at most 8 years, at most 9 years, at most 10 years, at most 11 years, or at most 12 years.

As described above, the biological sample used in methods described herein can include tissue (e.g., a tissue biopsy, surgically resected tissue) from a tumor. The tumor can be a tumor previously removed from the individual at risk for incidence or recurrence of cancer who is a cancer survivor. The biological sample can include both a tissue from a tumor and another biological sample from the individual. For example, tissue from a tumor and a liquid biopsy sample can be collected from the cancer survivor and analyzed by methods described herein to assess if the individual has a cancer recurrence (e.g., a relapse of a cancer). In some embodiments, the individual at risk for incidence or recurrence of cancer is diagnosed with cancer of unknown origin (e.g., diagnosed by methods described herein, diagnosed prior to practice of methods described herein, diagnosed following the practice of methods described herein).

The individual at risk for incidence or recurrence of cancer can have no prior history of cancer (e.g., an individual with no prior cancer diagnosis, the individual is not a cancer survivor). An individual at risk for incidence or recurrence of cancer can have undetectable cancer, a cancer that is not currently detectable, or undiagnosed cancer. In some embodiments, no tumor has been located in the individual at risk for incidence or recurrence of cancer. For example, no tumor has been located prior to, during, and/or following the practice of methods described herein. The individual can be identified as an individual at risk for incidence or recurrence of cancer by a risk stratification parameter as described above and analysis of a biological sample from the individual by an MCD test can lead to a positive result, but no cancer has been identified in the individual (e.g., no tumor, no cancer diagnosis). Such individuals can be termed “unresolved positives.” In some embodiments, the individual at risk for incidence or recurrence of cancer has not yet been diagnosed with a cancer prior to practicing of methods described herein. In some embodiments, the individual at risk for incidence or recurrence of cancer has not yet been diagnosed with a cancer prior to the step of analyzing the biological sample of methods described herein. In some embodiments, the individual at risk for incidence or recurrence of cancer has not yet been diagnosed with a cancer prior to the step of identifying the individual at risk for incidence or recurrence of cancer based on a risk stratification parameter. In some embodiments, the individual at risk for incidence or recurrence of cancer has not yet been diagnosed with a cancer prior to the step of based on the MCD test result (e.g., a positive MCD test result) and optionally the risk stratification parameter, administering a neoantigen immunogenic composition to the individual at risk for incidence or recurrence of cancer in need thereof.

The individual at risk for incidence or recurrence of cancer can be diagnosed with or can have a pre-cancer or a benign cancer (e.g., pre-cancer colorectal polyps, monoclonal gammopathy of unknown significance (MGUS), detection of a genetic variant of uncertain significance (VUS)). The individual can be diagnosed with or have a pre-cancer or a benign cancer prior to any of the steps of methods described herein (e.g., a) identifying the individual at risk for incidence or recurrence of cancer based on a risk stratification parameter, b) analyzing a biological sample from the individual at risk for incidence or recurrence of cancer using a multi-cancer detection (MCD) test to yield an MCD test result, and c) based on the MCD test result and optionally the risk stratification parameter, administering a neoantigen immunogenic composition to the individual at risk for incidence or recurrence of cancer in need thereof).

The individual at risk for incidence or recurrence of cancer can be diagnosed with any stage or grade of cancer. The individual at risk for incidence or recurrence of cancer can be diagnosed with cancer that is stage 0, I, II, III, or IV. The individual at risk for incidence or recurrence of cancer can be diagnosed with a cancer that is grade X, 1, 2, 3, or 4. The diagnosis of cancer grade or stage can be prior to, concurrently with, or following practice of methods described herein or any steps of the methods. The individual at risk for incidence or recurrence of cancer can be diagnosed with an early stage cancer, such as stage 0 cancer, stage I cancer, or carcinoma in-situ. As described below, early stage cancer diagnoses frequently result in no treatment by conventional standards of care (CSOC). It is believed, without out wishing to be bound by any particular mechanism of action or theory, that methods described herein are low in side effects and can be practiced on individuals with early stage cancer diagnoses that would otherwise, under the conventional standard of care (CSOC), receive no treatment (e.g., “watchful waiting”, “watch and wait”). The individual at risk for incidence or recurrence of cancer can be diagnosed with a slow growing cancer (e.g., a cancer type that is known to grow slowly). The individual at risk for incidence or recurrence of cancer can elect to hold off, delay, or forego conventional cancer treatments (e.g., surgery, chemotherapy, radiation, an immunotherapy, a target therapy, a therapy that is not a neoantigen immunogenic composition of methods described herein).

The individual can be diagnosed with any type of cancer. In some embodiments, the cancer is a type of cancer that the conventional standard of care (CSOC) is no treatment (e.g., “watchful waiting,” “watch and wait”). The cancer can be any type of cancerous tumor, including hematological malignancies, solid tumors, sarcomas, carcinomas, and other solid and non-solid tumors. Illustrative suitable cancers include, for example, adrenocortical carcinoma, anal cancer, appendiceal cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemias (e.g., acute lymphoblastic leukemia, acute myeloid leukemia, chronic lymphocytic leukemia, chronic myelogenous leukemia, hairy cell leukemia, myelodysplastic syndrome, prolymphocytic leukemia, large granular lymphocytic leukemia, adult T-cell leukemia, clonal eosinophilias), lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sézary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, or Wilms tumor. In some embodiments the individual at risk for incidence or recurrence of cancer is diagnosed with a cancer that is chronic lymphocytic leukemia, ductal carcinoma in situ (DCIS), hormone receptor-positive breast cancer, non-Hodgkin's lymphoma, prostate cancer, or rectal cancer.

### Conventional Standard of Care

Methods described herein can include the step of based on the MCD test result (e.g., a positive MCD test result) and optionally the risk stratification parameter, administering a neoantigen immunogenic composition to the individual at risk for incidence or recurrence of cancer in need thereof. It is believed, without wishing to be bound by any particular mechanism of action or theory, that neoantigen immunogenic compositions administered in methods described herein can be without or low in side effects compared to conventional standard of care (CSOC) therapies and procedures. It is further believed that such methods can be practiced on individuals with early cancer diagnoses that receive no treatment (e.g., no curative treatment) under the conventional standard of care. As used herein, “conventional standard of care” (CSOC) refers to any standard of care treatment for an individual for a given cancer diagnosis (e.g., a stage of cancer diagnosis, a type of cancer diagnosis, a cancer diagnosis given the health condition or age of the individual) excluding methods and neoantigen immunogenic compositions (e.g., cancer vaccines) described herein. The conventional standard of care can be any type of care including but not limited to surgery, radiation, chemotherapy, immunotherapy, targeted therapy, hormone therapy, photodynamic therapy, cell therapy, or a combination of any of the foregoing. Following certain diagnoses (e.g., slow growing cancers, cancers without a conventional efficacious treatment, early stage cancers, cancers in individuals where side effects of treatments are deemed intolerable, minimal residual disease (MRD)), the conventional standard of care is no treatment (e.g., “watch and wait,” “active surveillance,” or “watchful waiting” conventional standard of care). The conventional standard of care can be determined prior to, concurrent with, as a result of, or following practice of any method described herein. In some embodiments, the conventional standard of care (CSOC) for an individual at risk for incidence or recurrence of cancer at the time of practicing the method is no treatment. In some embodiments, the conventional standard of care for an individual at risk for incidence or recurrence of cancer does not include surgery, radiation, chemotherapy, immunotherapy, targeted therapy, hormone therapy, photodynamic therapy, cell therapy, or a combination of any of the foregoing.

### Efficacy of Methods

The methods described herein can be efficacious in treating an individual at risk for incidence or recurrence of cancer in need thereof. The methods can prevent cancer to any extent, such as prevent the incidence of cancer (e.g., prevent a first cancer diagnosis in an individual at risk for incidence or recurrence of cancer), prevent the relapse or recurrence of a cancer (e.g., prevent cancer relapse or recurrence in a cancer survivor), prevent a form of cancer (e.g., prevent an aggressive cancer, formation or development of an advanced stage of cancer, prevent metastasis of a cancer), or a combination of any of the foregoing. The methods can prevent the progression of an early stage cancer (e.g., a stage 0 cancer, a stage I cancer, VUS, MGUS), an in-situ carcinoma, a benign cancer, or a pre-cancer to an advanced stage of cancer (e.g., stage II cancer, stage III cancer, stage IV cancer). The methods can prevent the formation of a detectable cancer or tumor in an individual. The methods can prevent the metastasis of a cancer (e.g., the metastasis of a primary tumor to secondary tumor(s)). The methods can prevent a cancer from spreading from a site of primary tumor origin to other bodily sites in the individual.

The efficacy of methods described herein can be evaluated (e.g., determined) by any method. For example, the efficacy can be determined by assessing the disease-free (e.g., cancer-free) time period of an individual following practicing the method (e.g., a time period without cancer of at least 1 month, at least 2 months, at least 3 months, at least 4 months, at least 5 months, at least 6 months, at least 7 months, at least 8 months, at least 9 months, at least 10 months, at least 11 months, at least 1 year, at least 2 years, at least 3 year, at least 4 years, at least 5 years, at least 6 years, at least 7 years, at least 8 years, at least 9 years, at least 10 years, at least 11 years, at least 12 years, or more). The efficacy can be determined by the identification or lack thereof of a tumor or diagnosis of a cancer in the individual. The efficacy can be determined by the survival of the individual (e.g., the survival of at least 1 month, at least 2 months, at least 3 months, at least 4 months, at least 5 months, at least 6 months, at least 7 months, at least 8 months, at least 9 months, at least 10 months, at least 11 months, at least 1 year, at least 2 years, at least 3 year, at least 4 years, at least 5 years, at least 6 years, at least 7 years, at least 8 years, at least 9 years, at least 10 years, at least 11 years, at least 12 years, or more).

One method of determining efficacy of the method can be by analyzing one or more subsequent biological samples from the individual using a second multi-cancer detection (MCD) test (e.g., a second MCD test administered during the course of treatment of the individual). Any number of subsequent biological samples can be analyzed by an MCD test including but not limited to 1, 2, 3, 4, 5, 6, 7, 8, 9, 10, 11, 12, 13, 14, 15, 16, 17, 18, 19, or 20. The number of subsequent biological samples analyzed can be at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20. The number of subsequent biological samples analyzed can be between about 1 to about 10, about 1 to about 9, about 1 to about 8, about 1 to about 7, about 1 to about 6, about 1 to about 5, about 1 to about 4, about 1 to about 3, about 1 to about 2, about 2 to about 10, about 2 to about 9, about 2 to about 8, about 2 to about 7, about 2 to about 6, about 2 to about 5, about 2 to about 4, about 2 to about 3, about 3 to about 10, about 3 to about 9, about 3 to about 8, about 3 to about 7, about 3 to about 6, about 3 to about 5, about 3 to about 4, about 4 to about 10, about 4 to about 9, about 4 to about 8, about 4 to about 7, about 4 to about 6, about 4 to about 5, about 5 to about 10, about 5 to about 9, about 5 to about 8, about 5 to about 7, about 5 to about 6, about 6 to about 10, about 6 to about 9, about 6 to about 8, about 6 to about 7, about 7 to about 10, about 7 to about 9, about 7 to about 8, about 8 to about 10, about 8 to about 9, or about 9 to about 10. The second multi-cancer detection (MCD) test can be the same type (e.g., same manufacturer, same detection method, same analyte detected, same parameter of the sample detected, same chemistry) as a first MCD test used in the step of analyzing a biological sample from the individual at risk for incidence or recurrence of cancer in methods described herein. For example, the first MCD test and the second MCD test can both be sequencing of cell free DNA (cfDNA) from the blood of an individual. The second multi-cancer detection (MCD) test can be a different type (e.g., different manufacturer, different detection method, different analyte detected, different parameter of the sample detected, different chemistry) as a first MCD test used in the step of analyzing a biological sample from the individual at risk for incidence or recurrence of cancer in methods described herein. For example, the first MCD test can be sequencing of cell free DNA (cfDNA) from the blood of an individual and the second MCD test can be detection of a circulating cancer antigen by immunohistochemistry. Methods can comprise the step of analyzing a second biological sample from an individual at risk for incidence or recurrence of cancer using a second multi-cancer detection (MCD) test (e.g., during the course of treatment) to determine efficacy of the neoantigen immunogenic composition. The step of analyzing a second biological sample using a second multi-cancer detection (MCD) test can be repeated any number of times over the course of treatment for the individual at risk for incidence or recurrence of cancer for different subsequent biological samples. The step of analyzing a second biological sample can be repeated any number of times, including but not limited to 1, 2, 3, 4, 5, 6, 7, 8, 9, 10, 11, 12, 13, 14, 15, 16, 17, 18, 19, or 20 times. The number repetitions can be at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20. The number of repetitions can be between about 1 to about 10, about 1 to about 9, about 1 to about 8, about 1 to about 7, about 1 to about 6, about 1 to about 5, about 1 to about 4, about 1 to about 3, about 1 to about 2, about 2 to about 10, about 2 to about 9, about 2 to about 8, about 2 to about 7, about 2 to about 6, about 2 to about 5, about 2 to about 4, about 2 to about 3, about 3 to about 10, about 3 to about 9, about 3 to about 8, about 3 to about 7, about 3 to about 6, about 3 to about 5, about 3 to about 4, about 4 to about 10, about 4 to about 9, about 4 to about 8, about 4 to about 7, about 4 to about 6, about 4 to about 5, about 5 to about 10, about 5 to about 9, about 5 to about 8, about 5 to about 7, about 5 to about 6, about 6 to about 10, about 6 to about 9, about 6 to about 8, about 6 to about 7, about 7 to about 10, about 7 to about 9, about 7 to about 8, about 8 to about 10, about 8 to about 9, or about 9 to about 10. A negative test result for the second or subsequent MCD test can indicate the method and/or neoantigen immunogenic composition (e.g., the cancer vaccine) is efficacious. A positive test result for the second or subsequent MCD test can indicate the method and/or neoantigen immunogenic composition is not yet efficacious. A positive test result can indicate that any of the steps of the method must be repeated (e.g., repetition of analyzing a biological sample from the individual at risk for incidence or recurrence of cancer using a multi-cancer detection (MCD) test to yield an MCD test result; repetition of based on the MCD test result and optionally the risk stratification parameter; administering a neoantigen immunogenic composition to the individual at risk for incidence or recurrence of cancer in need thereof; repetition of analyzing a second biological sample from the individual at risk for incidence or recurrence of cancer using a second multi-cancer detection (MCD) test (e.g., during the course of treatment) to determine efficacy of the neoantigen immunogenic composition).

The efficacy of methods described herein can be evaluated by analyzing one or more specific biomarkers. The specific biomarkers can be any biomarkers present in a biological sample from the individual at risk for incidence or recurrence of cancer including, but not limited to a nucleic acid (e.g., a nucleic acid sequence, RNA, DNA), a cancer antigen, an extracellular vesicle protein, a neoantigen peptide, and combinations of any of the foregoing. The specific biomarker can be identified by the MCD test (e.g., a sequence variation in a nucleic acid identified by an MCD test).

### Co-Therapies

Methods described herein can include administering a co-therapy or procedure in addition to a neoantigen immunogenic composition (e.g., a cancer vaccine). Without wishing to be bound by any particular mechanism of action or theory, it is believed that administration of neoantigen immunogenic compositions by methods described herein can deescalate or reduce the frequency, intensity, or dosage of a co-therapy or procedure (e.g., a conventional standard of care therapy) while maintaining the efficacy of the co-therapy or procedure in comparison to the co-therapy or procedure alone (e.g., the co-therapy or procedure without administration of a neoantigen immunogenic composition as described herein). For example, the dosage of a chemotherapeutic agent can be reduced while maintaining efficacy of the chemotherapeutic by administering to an individual at risk for incidence or recurrence of cancer a neoantigen immunogenic composition in methods described herein.

The co-therapy or procedure can be any co-therapy or procedure, including any therapeutic agents and procedures for treating a neoplastic disease (e.g., a cancer). In certain embodiments, the co-therapy or procedure is not a neoantigen immunogenic composition (e.g., is not a cancer vaccine, is not a neoantigen immunogenic composition described herein). In certain embodiments, the co-therapy or procedure is a radiation treatment, a surgical procedure, a chemotherapeutic, an immunotherapy, a targeted therapy, a hormone therapy, a photodynamic therapy, a cell therapy, or a combination of any of the foregoing. In certain embodiments, the step of administering a neoantigen immunogenic composition to an individual at risk for incidence or recurrence of cancer in need thereof further includes administering a co-therapy or procedure that (i) is not the neoantigen immunogenic composition and (ii) is a radiation treatment, a surgical procedure, a chemotherapeutic, an immunotherapy, a targeted therapy, a hormone therapy, a photodynamic therapy, a cell therapy, or a combination of any of the foregoing. The radiation treatment can be any radiation therapy including but not limited to external beam radiation therapy (e.g., image-guided radiation therapy (IGRT), Tomotherapy, stereotactic radiosurgery, stereotactic body radiation therapy, photon external beam radiation therapy, photon external beam radiation therapy, proton external beam radiation therapy, electron external beam radiation therapy), internal radiation therapy (e.g., brachytherapy, systemic therapy), or combinations of any of the foregoing. The surgical procedure can be any surgical procedure including, but not limited to curative surgery (e.g., removal of a tumor), preventative surgery (e.g., removal of precancerous tissue), diagnostic surgery (e.g., removal of a tissue sample), staging surgery, restorative surgery (e.g., cosmetic surgery following cancer treatment) or combinations of any of the foregoing.

An immunotherapy administered in methods described herein can contain any immunotherapeutic agent including, but not limited to, immune checkpoint inhibitors (e.g., abatacept, atezolizumab, avelumab, cemiplimab, durvalumab, ipilimumab, nivolumab, pembrolizumab, spartalizumab, tremelimumab), chimeric antigen receptor cell therapy (e.g., chimeric antigen receptor (CAR) T-cell therapy, CAR-macrophage therapy, CAR-natural killer cell therapy), cancer vaccine, adalimumab, anakinra, dupilumab, etanercept, mepolizumab, omalizumab, rituximab, secukinumab, tumor infiltrating lymphocyte (TIL) therapy, tocilizumab, ustekinumab, and combinations thereof. Immune checkpoint inhibitors can bind and/or inhibit any inhibitory checkpoint molecule including, but not limited to, adenosine A2A receptor, adenosine A2B receptor, B7-H3, B7-H4, B and T lymphocyte attenuator, cytotoxic T-lymphocyte-associated protein 4, indoleamine 2,3-dioxygenase, killer-cell immunoglobulin-like receptor, lymphocyte activation gene-3, nicotinamide adenine dinucleotide phosphate NADPH oxidase isoform 2, programmed cell death 1 protein (PD-1), programmed death ligand 1 (PD-L1), programmed death ligand 2 (PD-L2), T-cell immunoglobulin domain and mucin domain 3, galectin 9, V-domain Ig suppressor of T cell activation, sialic acid-binding immunoglobulin-type lectin 7, sialic acid-binding immunoglobulin-type lectin 9, and combinations thereof. Immunotherapies can also bind to or activate stimulatory immune checkpoint molecules including, but not limited to, CD27, CD28, CD40, CD122, CD137, OX40, glucocorticoid-induced TNFR family related gene, inducible T-cell costimulator, and combinations thereof.

Targeted therapies treat cancer by interrupting unique molecular abnormalities that drive cancer growth. Targeted therapeutics are designed to interfere with a specific biochemical pathway central to the development, growth, and/or spread of a particular cancer. Typically, a targeted therapy is selected and administered based on mutations present in an individual's cancer or tumor. Methods disclosed herein can administer any targeted therapy to an individual, including but not limited to abemaciclib, acalabrutinib, adagrasib, ado-trastuzumab emtansine, afatinib dimaleate, alectinib, alemtuzumab, alpelisib, amivantamab-vmjw, anastrozole, asciminib hydrochloride, atezolizumab, avapritinib, avelumab, axitinib, belzutifan, bevacizumab, binimetinib, blinatumomab, bosutinib, brexucabtagene autoleucel, brigatinib, cabozantinib-s-malate, capmatinib hydrochloride, capivasertib, cemiplimab-rwlc, ceritinib, cetuximab, crizotinib, dabrafenib, dacomitinib, dasatinib, denosumab, durvalumab, duvelisib, elacestrant dihydrochloride, enasidenib mesylate, encorafenib, enfortumab vedotin-ejfv, entrectinib, erdafitinib, erlotinib, everolimus, exemestane, fam-trastuzumab deruxtecan-nxki, fruquintinib, fulvestrant, futibatinib, gefitinib, gemtuzumab ozogamicin, gilteritinib fumarate, glasdegib maleate, ibrutinib, idelalisib, imatinib, infigratinib phosphate, inotuzumab ozogamicin, iobenguane I 131, ipilimumab, ivosidenib, lanreotide acetate, lapatinib ditosylate, 57envatinib mesylate, letrozole, lorlatinib, lutetium Lu 177-dotatate, margetuximab-cmkb, midostaurin, moxetumomab pasudotox-tdfk, cecitumumab, neratinib maleate, nilotinib, nivolumab, obinutuzumab, ofatumamb, olaparib, olutasidenib, osimertinib mesylate, palbociclib, panitumumab, pazopanib hydrochloride, pembrolizumab, pemigatinib, pertuzumab, pexidartinib hydrochloride, pirtobrutinib, ponatinib hydrochloride, pralsetinib, quizartinib dihydrochloride, ramucirumab, repotrectinib, regorafenib, ribociclib, ripretinib, rituximab, sacituzumab govitecan-hziy, selpercatinib, sorafenib tosylate, sotorasib, sunitinib malate, tagraxofusp-erzs, talazoparib tosylate, tamoxifen citrate, temsirolimus, tepotinib hydrochloride, tisagenlecleucel, tisotumab vedotin-tftv, tivozanib hydrochloride, toremifene, toripalimab-tpzi, trametinib, trastuzumab, tremelimumab-actl, tretinoin, tucatinib, vemurafenib, venetoclax, zanubrutinib, ziv-aflibercept, or combinations of any of the foregoing.

A neoantigen immunogenic composition described herein can be administered to an individual in methods described herein alone or in combination with other therapeutic agents. Any suitable chemotherapeutic treatment for a particular cancer can be administered in methods described herein. Exemplary chemotherapeutic agents include, but are not limited to aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol®), pilocarpine, prochlorperazine, rituximab, tamoxifen, topotecan hydrochloride, trastuzumab, vinblastine, vincristine and vinorelbine tartrate. The individual may be administered a small molecule, or targeted therapy (e.g., kinase inhibitor).

The co-therapy can be any hormone therapy. Suitable hormone therapies include but are not limited to aromatase inhibitors (e.g., anastrozole, exemestane, letrozole), selective estrogen receptor modulators (SERMs) (e.g., tamoxifen, raloxifene), estrogen receptor antagonists (e.g., fulvestrant, toremifene), luteinizing hormone-releasing hormone (LHRH) agonists (e.g., goserelin, leuprolide, triptorelin, degarelix), anti-androgens (e.g., apalutamide, enzalutamide, darolutamide, bicalutamide, flutamide, nilutamide), CYP17 inhibitors (e.g., biraterone, ketoconazole), progestins (e.g., medroxyprogesterone acetate, megestrol acetate), adrenolytics (e.g., mitotane), or combinations of any of the foregoing.

### EQUIVALENTS

It will be readily apparent to those skilled in the art that other suitable modifications and adaptions of the methods of the invention described herein are obvious and may be made using suitable equivalents without departing from the scope of the disclosure or the embodiments. Having now described certain compositions and methods in detail, the same will be more clearly understood by reference to the following example, which is introduced for illustration only and not intended to be limiting.

### Example

### Example 1. Use of an MCD Test to Select an Individual with a Positive Early Cancer Signal for Curative Cancer Vaccine Treatment

An adult individual who has an elevated risk of developing breast or ovarian cancer will be identified based on family history of cancer incidence in related individuals and being a carrier of an inherited (germline) BRCA1 mutation. The individual will have not yet received a cancer diagnosis (i.e., the individual will never have been diagnosed with cancer) but will have recently taken a GRAIL Galleri® multi-cancer test (GRAIL, LLC; ILLUMINA, INC.), which is a type of MCD test capable of measuring likely cancer signals based on circulating tumor DNA. About 2 weeks following administration of the Galleri® test, results from the Galleri® test will show a positive cancer signal in the blood, along with Tissue of Origin (TOO) that will indicate that the signal is likely originating from an ovary or uterus.

Upon receiving the positive MCD test result, the individual will undergo a series of CT scans focusing on the ovaries and uterus but no tumor will be detected at the time of scan. The individual will then be advised by the care team to monitor symptoms and progression of the cancer before treatment can occur, which is a “watchful waiting” strategy (i.e., there will be no standard of care cancer treatment prescribed). However, the individual will continue to worry about the chance of tumor progression and will consider treatment options that may include preemptive removal of one or both ovaries. In this scenario, a cancer vaccine will be administered when a “watchful waiting” strategy would otherwise be prescribed and will have few or no known side effects compared to other more aggressive forms of treatment.

The individual will be selected for potential cancer vaccine treatment with the intent to cure the early cancer, pending the discovery of targetable mutations from the circulating tumor DNA. After receiving the initial positive MCD test, a follow up ‘intensified’ sequencing assay will be ordered for the individual, requesting additional blood samples to be collected to improve the sensitivity and precision of finding tumor mutations suitable for targeting with a vaccine.

The intensified sequencing assay will be based on ultra-deep whole exome sequencing (WES) to look for rare tumor mutations that induce protein-coding changes. Using only blood from the individual, both cell-free DNA in plasma (for tumor signal) and genomic DNA in the buffy coat (primarily white blood cells; for normal signal as background) will be sequenced. Additionally, cell-free RNA in plasma may also be sequenced for additional evidence that the mutations are expressed. To further increase the precision, error suppressive techniques such as Unique Molecular Identifiers (UMI) may be used during sequencing. The assay may combine repeated sampling of blood, if needed, to obtain high quality results. The sequencing results, including plasma and buffy coat DNA and optionally RNA data, will be delivered between about 1 week to about 2 weeks following blood collection.

The set of variants that are specifically represented in the plasma cell-free DNA, but not in the buffy coat DNA, will be identified bioinformatically. Custom tumor-normal (T/N) pipelines, e.g., Illumina DRAGEN Bio-IT solution, will be used to facilitate the variant calling. Germline variants and common aging-related variants in the blood (e.g., clonal hematopoiesis of indeterminate potential (CHIP, see for example M. M. Uddin, et al. Clonal hematopoiesis of indeterminate potential, DNA methylation, and risk for coronary artery disease. Nat. Commun. (2022), 13, 5350, 1-16)) will be subtracted from the set of candidate variants. False positive variant calls will be filtered to account for site-specific noise from sequencing. Other filters, such as the number of supporting fragments, strand bias, position of mutation within the fragments, base qualities or mapping qualities, fragment lengths, fragment end motifs, and the like will be used to ensure that the variants are of high quality and are likely specific to the tumor. The filters and associated cutoff values will be determined based on a set of diagnosed cancer patients with available paired tissue and blood biopsy samples as training data. Following applying these stringent filters, between 2 to 100 variants that are predicted to yield mutated peptides will be selected for neoantigen predictions and ranking.

A set of mutated peptide neoantigens will be derived from the identified tumor mutations. The HLA types for the individual at risk will be inferred with at least 4-digit precision. The set of neoantigens will be ranked based on a number of factors, including predicted MHC-I/II immunogenicity using BigMHC (see WO2022/119885A1) along with the HLA types, overall expression level in RNA, allele fraction, clonality, probability of tumor-specificity, association with cancer driver genes, and the like. Up to a total of the 48 highest ranking peptides will be ordered for synthesis, which typically include 2 to 42 long peptides and 2 to 20 short peptides. About 4 to 6 weeks following the sequencing assay, a personalized cancer vaccine will be formulated by making 4 pools among the peptides that are successfully synthesized, with a total of up to 20 peptides across the pools. The aggregated immunogenicity of the vaccine is computed to be between 5% to 99%, which estimates the probability that the vaccine successfully mounts an oncolytic immune response.

The individual will follow a treatment schema with multiple vaccinations given consecutively at a regular interval over the course of several months, with doses selected to have previously shown no significant toxicity or related adverse events. The individual will also receive the adjuvant, such as Poly ICLC, both admixed with the peptide vaccine, and also separately given regularly to prime and boost anti-tumor T cell responses.

Clinical response of the individual will be monitored through follow-up Galleri® tests, at 6 and 12 months following the vaccination. Negative test results for the Galleri® tests will serve to confirm that the cancer vaccine is efficacious in clearing the cancer signal previously observed. Additional sequencing of blood samples at 6 or 12 months may be performed to further confirm that previous tumor mutations are no longer detected. The individual will be regarded as cancer-free upon the molecular clearance of the circulating cancer signal, and will be able to return to a normal cadence of MCD testing, such as annual testing, to monitor new cancer or recurrence of cancer.

### Example 2. Analysis of Neoantigens Present in Subjects with Positive Multi-Cancer Detection Tests and Confirmed Cancer Diagnosis

Adult subjects with elevated risk of cancer were administered a multi-cancer detection (MCD) test capable of measuring likely cancer signals based on circulating tumor DNA. Subjects who received a positive MCD test result and cancer diagnosis were selected for potential cancer vaccine treatment with the goal of treating the cancer, pending the discovery of targetable mutations from the circulating tumor DNA. Additional blood samples were collected to perform whole exome sequencing (WES), with increased sensitivity and precision. Plasma were collected from total blood stored in Streck tubes to stabilize the samples. Cell free DNA were extracted from plasma using commercial cfDNA extraction kit. Illumina sequencing libraries were generated using capture probes targeting the protein coding genes, adding adapters and barcodes to track the sample of origin. Finally, libraries were sequenced on a NovaSeq X sequencer.

The number of neoantigens identified was quantified from the variants called by analysis of the sequencing data using Illumina DRAGEN. Samples were collected from 27 subjects for sequencing and analysis. Of the 27 samples sequenced, six samples identified one or more neoantigen.

1. A method of treating an individual at risk for incidence or recurrence of cancer in need thereof, comprising the steps of
a) identifying the individual at risk for incidence or recurrence of cancer based on a risk stratification parameter,
b) analyzing a biological sample from the individual at risk for incidence or recurrence of cancer using a multi-cancer detection (MCD) test to yield an MCD test result, and
c) based on the MCD test result and optionally the risk stratification parameter, administering a neoantigen immunogenic composition to the individual at risk for incidence or recurrence of cancer in need thereof.

2. The method of claim 1, wherein the risk stratification parameter is age, sex, family medical history, medications, germ-line mutations, somatic mutations, exposure to carcinogens, exposure to radiation, medical history, previous cancer diagnosis, or a combination of any of the foregoing.

3. The method of claim 1, wherein the biological sample from the individual at risk for incidence or recurrence of cancer is an amniotic fluid sample, an ascitic fluid sample, a blood sample, a buccal sample, a cerebrospinal fluid sample, a fecal sample, a hair sample, a peritoneal fluid sample, a pleural effusion sample, a saliva sample, a semen sample, a synovial fluid sample, a tissue sample, a urine sample, or a combination of any of the foregoing.

4. The method of claim 1, wherein the biological sample from the individual at risk for incidence or recurrence of cancer is a blood sample.

5. The method of claim 1, wherein the biological sample is a tissue sample from a tissue biopsy of a tumor or surgical tissue resection of a tumor.

6. The method of claim 1, wherein the MCD test analyzes nucleic acid methylation, nucleic acid mutation, nucleic acid fragmentation patterns, extracellular vesicle proteins, nucleic acid of circulating tumor cells, gene expression, circulating cancer antigens, or a combination of any of the foregoing.

7. The method of claim 1, wherein the MCD test comprises nucleic acid sequencing of the biological sample from the individual at risk for incidence or recurrence of cancer.

8. The method of claim 1, wherein the MCD test analyzes a nucleic acid that is cell free DNA (cfDNA), circulating tumor DNA (ctDNA), cell free RNA (cfRNA), circulating tumor RNA (ctRNA), or a combination of any of the foregoing.

9. The method of claim 1, further comprising the step of
sequencing and analyzing of the biological sample from the individual at risk for incidence or recurrence of cancer or a new biological sample from the individual at risk for incidence or recurrence of cancer to identify neoantigens to be included in the neoantigen immunogenic composition.

10. The method of claim 1, further comprising the steps of
determining the HLA type for the individual at risk for incidence or recurrence of cancer,
scoring neoantigens of the biological sample from the individual at risk for incidence or recurrence of cancer,
selecting neoantigens of the biological sample for inclusion in the neoantigen immunogenic composition, and/or
generating the neoantigen immunogenic composition.

11. The method of claim 1, wherein the individual at risk for incidence or recurrence of cancer is a cancer survivor.

12. The method of claim 1, wherein the conventional standard of care for the individual at risk for incidence or recurrence of cancer at the time of practicing the method is treated with conventional standard of care, wherein the conventional standard of care is no treatment.

13. The method of claim 1, wherein the individual at risk for incidence or recurrence of cancer has not been diagnosed with a cancer prior to the step of analyzing the biological sample.

14. The method of claim 1, wherein the individual at risk for incidence or recurrence of cancer has no identified tumor.

15. The method of claim 1, wherein the individual at risk for incidence or recurrence of cancer is diagnosed with cancer of unknown origin.

16. The method of claim 1, wherein the individual at risk for incidence or recurrence of cancer is treated with conventional standard of care, wherein the conventional standard of care does not include surgery, radiation, chemotherapy, immunotherapy, targeted therapy, hormone therapy, photodynamic therapy, cell therapy, or a combination of any of the foregoing.

17. The method of claim 1, further comprising the step of
analyzing a second biological sample from the individual at risk for incidence or recurrence of cancer using a second multi-cancer detection (MCD) test during a course of treatment to determine efficacy of the neoantigen immunogenic composition.

18. The method of claim 17, wherein a negative test result for the second MCD test indicates the neoantigen immunogenic composition is efficacious.

19. The method of claim 1, wherein step b) and optionally step c) are repeated at least once over a course of treatment for the individual at risk for incidence or recurrence of cancer.

20. The method of claim 1, wherein the individual at risk for incidence or recurrence of cancer is diagnosed with a cancer that is chronic lymphocytic leukemia, ductal carcinoma in situ (DCIS), hormone receptor-positive breast cancer, non-Hodgkin's lymphoma, prostate cancer, or rectal cancer.

21. The method of claim 1, wherein the individual at risk for incidence or recurrence of cancer is diagnosed with stage 0 cancer, stage I cancer, or carcinoma in-situ.

22. The method of claim 1, wherein the individual at risk for incidence or recurrence of cancer is diagnosed with a pre-cancer or a benign cancer.

23. The method of claim 1, wherein the method further comprises the step of sequencing nucleic acids of the biological sample to yield non-cancer sequencing results and cancer sequencing results, wherein the non-cancer sequencing results are subtracted from the cancer sequencing results to improve the signal to noise ratio for variant calling of neoantigens.

24. The method of claim 1, wherein the method further comprises administering a co-therapy or procedure that
(i) is not the neoantigen immunogenic composition and
(ii) is a radiation treatment, a surgical procedure, a chemotherapeutic, an immunotherapy, a targeted therapy, a hormone therapy, a photodynamic therapy, a cell therapy, or a combination of any of the foregoing.
